(19) United States
(12) Patent Application Publication
(10) Pub. No.: US 2025/0170152 A1
(43) Pub. Date: May 29, 2025

(54) Cannabinoid Dosage Form

(71) Applicant: Emyria, Leederville (AU)

(72) Inventors: Michael Winlo, Nedlands (AU); Tatjana Heinrich, Mount Pleasant (AU); Tracie Ernenwein, North Perth (AU); Bill Bosch, Osborne Park (AU); Sebastian Roth, Crawley (AU)

(21) Appl. No.: 18/841,901

(22) PCT Filed: Feb. 24, 2023
(86) PCT No.: PCT/AU2023/050129

§ 371 Date: Aug. 27, 2024

(30) Foreign Application Priority Data
Feb. 28, 2022 (AU) 2022900479

(51) Int. Cl. A61K 31/00; A61K 9/00; A61K 9/48; A61K 47/10

(52) U.S. Cl. CPC A61K 31/658; A61K 9/0056; A61K 9/4858; A61K 47/10

(57) ABSTRACT

A pharmaceutical composition comprising a cannabinoid; and a polyethylene glycol (PEG) ester.

### TECHNICAL FIELD

The present invention relates to a pharmaceutical composition for the delivery of a cannabinoid, such as cannabidiol.

### BACKGROUND ART

There is increasing interest in the use of cannabinoids for disease and symptom management, but limited information available regarding their pharmacokinetics and pharmacodynamics to guide prescribers.

Many Cannabis medicines, particularly those based on plant extracts, contain a wide variety of chemical compounds, including the cannabinoids delta-9-tetrahydrocannabinol (THC), which is psychoactive, and the nonpsychoactive cannabidiol (CBD). CBD has been reported to have analgesic, neuroprotective, anticonvulsant, antiemetic, antispasmodic and anti-inflammatory properties.

A variety of standardized, medical-grade Cannabis plant-derived or synthetically produced cannabinoid products have been developed for medicinal use. By contrast, nonmedical-grade products are nonstandardized and contain unknown amounts of THC and CBD.

Oromucosal preparations [e.g. Sativex™ (nabiximols) oromucosal spray] undergo rapid absorption via the oral mucosa (and hence are useful for symptoms requiring rapid relief), producing plasma drug concentrations higher relative to oral, but reduced relative to inhaled THC. However, part of the dose may be swallowed and orally absorbed.

THC and CBD are both highly lipophilic and have poor oral bioavailability (estimated to be as low as 6%). Oral THC formulations exhibit variable absorption and undergo extensive hepatic first-pass metabolism, resulting in lower peak plasma THC concentration relative to inhalation and a longer delay (˜120 min) to reach peak concentration. Following oral administration of CBD, a similar plasma concentration-time profile to that of oral THC has been observed. Based on this profile, oral formulations may be useful for patients requiring symptomatic relief over a longer period.

However, many oral pharmaceutical compositions have been associated with gastrointestinal side effects. Furthermore, the oil-based liquid compositions are inconvenient to dose, and dispensing may be messy due to their oil-based nature.

There is a need to develop novel cannabinoid compositions; or at least the provision of cannabinoid compositions to compliment the previously known cannabinoid delivery methods. The present invention seeks to provide an improved or alternative cannabinoid compositions, preferably compositions that have increased bioavailability.

The previous discussion of the background art is intended to facilitate an understanding of the present invention only. The discussion is not an acknowledgement or admission that any of the material referred to is or was part of the common general knowledge as at the priority date of the application.

### SUMMARY OF INVENTION

The present invention provides a pharmaceutical composition comprising:
a) a cannabinoid; and
b) a polyethylene glycol (PEG) ester.

Preferably the pharmaceutical composition does not compromise an oil or lipid, or only compromises a minimal amount of an oil or lipid.

Preferably the pharmaceutical composition comprises a ratio of from 1:5 w/w cannabinoid:PEG ester to 2:3 w/w cannabinoid:PEG ester.

The present invention provides a pharmaceutical composition comprising:
a) a cannabinoid; and
b) a polyethylene glycol (PEG) ester
   wherein the bioavailability of the pharmaceutical composition comprising a polyethylene glycol (PEG) ester is from 0.8 to 12 times higher than the bioavailability of an oral pharmaceutical composition which comprises the same amount of cannabinoid but does not comprise a polyethylene glycol (PEG) ester.

The present invention further provides a pharmaceutical composition comprising:
a) a cannabinoid; and
b) a polyethylene glycol (PEG) ester
   wherein the prophylactically or therapeutically effective amount of the pharmaceutical composition comprising a polyethylene glycol (PEG) ester is from 0.08 to 1.25 times the prophylactically or therapeutically effective amount of an oral pharmaceutical composition which comprises the same amount of cannabinoid but does not comprise a polyethylene glycol (PEG) ester.

Preferably the bioavailability is measured using:
i. AUC₀₋₂₄ and the AUC₀₋₂₄ of a cannabinoid in a composition of the present invention is from 0.8 to 12 times higher compared to the AUC₀₋₂₄ of a cannabinoid in a composition that does not comprise a PEG ester;
ii. Tₘₐₓ and a cannabinoid in a composition of the present invention reaches the same Tₘₐₓ as cannabinoid in a composition that does not comprise a PEG ester from 0.5 to 2 times earlier than the Tₘₐₓ of the comparator composition;
iii. Cₘₐₓ and the Cₘₐₓ of a cannabinoid in a composition of the present invention is from 0.8 to 12 times higher compared to the Cₘₐₓ of a cannabinoid in a composition that does not comprise a PEG ester;
iv. AUC_{0-inf} and the AUC_{0-inf} of a cannabinoid in a composition of the present invention is from 0.8 to 12 times higher compared to the AUC_{0-inf} of a cannabinoid in a composition that does not comprise a PEG ester; and/or
v. AUCₗₐₛₜ and the AUCₗₐₛₜ of a cannabinoid in a composition of the present invention is from 0.8 to 12 times higher compared to the AUCₗₐₛₜ of a cannabinoid in a composition that does not comprise a PEG ester
when the same dose of cannabinoid is provided in the present composition and the comparator composition.

Preferably a cannabinoid in a composition of the present invention has:
i. an AUC₀₋₂₄ of from 250 hr*ng/mL to 2000 hr*ng/mL;
ii. a Tₘₐₓ of from 1.2 h to 8 h;
iii. a Cₘₐₓ of from 40 ng/mL to 400 ng/mL;
iv. an AUC_{0-inf} of from 250 hr*ng/mL to 2,000 hr*ng/mL; and/or
v. an AUCₗₐₛₜ of from 250 hr*ng/mL to 2,000 hr*ng/mL
   measured after administration of a dose of from 1 mg/kg/day to 25 mg/kg/day of a cannabinoid.

Preferably a cannabinoid in a composition of the present invention has:
i. an AUC₀₋₂₄ of from 197.9% to 795.3% of a cannabinoid in a composition that does not comprise a PEG ester;
ii. a Cₘₐₓ of from 34.0% to 247.3% of a cannabinoid in a composition that does not comprise a PEG ester;
iii. an AUC_{0-inf} of from 208.0% to 806.2% of a cannabinoid in a composition that does not comprise a PEG ester; and/or
iv. an AUCₗₐₛₜ of from 197.9% to 795.3% of a cannabinoid in a composition that does not comprise a PEG ester
when the same dose of cannabinoid is provided in the present composition and the comparator composition.

Preferably the comparator composition that does not comprise a PEG ester is Epidyolex™.

Preferably the pharmaceutical composition is an oral pharmaceutical composition.

Preferably, the cannabinoid is chosen from the list comprising: cannabidiol, cannabinol, cannabigerol, cannabichromene, and Δ⁹-tetrahydrocannabinol. Most preferably, the cannabinoid is cannabidiol.

Preferably the polyethylene glycol (PEG) ester is a stearoyl or lauroyl macrogol-32 glyceride or vitamin E polyethylene glycol succinate.

The present invention further provides a method for the treatment, amelioration or prevention of a condition responsive to administration of a cannabinoid, said method comprising the step of:
a) administering a pharmaceutical composition comprising:
   i. a cannabinoid;
   ii. a polyethylene glycol (PEG) ester.

The present invention further provides a method for the treatment, amelioration or prevention of a condition responsive to administration of a cannabinoid, said method comprising the step of:
a) administering a pharmaceutical composition comprising:
   i. a cannabinoid;
   ii. a polyethylene glycol (PEG) ester
      wherein the bioavailability of the pharmaceutical composition comprising a polyethylene glycol (PEG) ester is from 0.8 to 12 times higher than the bioavailability of an oral pharmaceutical composition which comprises the same amount of cannabinoid but does not comprise a polyethylene glycol (PEG) ester.

The present invention further provides a method for the treatment, amelioration or prevention of a condition responsive to administration of a cannabinoid, said method comprising the step of:
a) administering a pharmaceutical composition comprising:
   i. a cannabinoid;
   ii. a polyethylene glycol (PEG) ester
      wherein the prophylactically or therapeutically effective amount of the pharmaceutical composition comprising a polyethylene glycol (PEG) ester is from 0.08 to 1.25 times the prophylactically or therapeutically effective amount of an oral pharmaceutical composition which comprises the same amount of cannabinoid but does not comprise a polyethylene glycol (PEG) ester.

The present invention provides for the use of:
a) a cannabinoid; and
b) a polyethylene glycol (PEG) ester
   for the manufacture of a pharmaceutical composition for the treatment, amelioration or prevention of a condition responsive to administration of a cannabinoid.

The present invention provides for the use of:
a) a cannabinoid; and
b) a polyethylene glycol (PEG) ester
   for the manufacture of a pharmaceutical composition for the treatment, amelioration or prevention of a condition responsive to administration of a cannabinoid, wherein the bioavailability of the pharmaceutical composition comprising a polyethylene glycol (PEG) ester is from 0.8 to 12 times higher than the bioavailability of an oral pharmaceutical composition which comprises the same amount of cannabinoid but does not comprise a polyethylene glycol (PEG) ester.

The present invention further provides for the use of:
a) a cannabinoid; and
b) a polyethylene glycol (PEG) ester
   for the manufacture of a pharmaceutical composition for the treatment, amelioration or prevention of a condition responsive to administration of a cannabinoid, wherein the prophylactically or therapeutically effective amount of the pharmaceutical composition comprising a polyethylene glycol (PEG) ester is from 0.08 to 1.25 times the prophylactically or therapeutically effective amount of an oral pharmaceutical composition which comprises the same amount of cannabinoid but does not comprise a polyethylene glycol (PEG).

The present invention provides for the use of a composition comprising:
a) a cannabinoid; and
b) a polyethylene glycol (PEG) ester
   for the treatment, amelioration or prevention of a condition responsive to administration of a cannabinoid.

The present invention further provides for the use of a composition comprising:
a) a cannabinoid; and
b) a polyethylene glycol (PEG) ester
   for the treatment, amelioration or prevention of a condition responsive to administration of a cannabinoid, wherein the bioavailability of the pharmaceutical composition comprising a polyethylene glycol (PEG) ester is from 0.8 to 12 times higher than the bioavailability of an oral pharmaceutical composition which comprises the same amount of cannabinoid but does not comprise a polyethylene glycol (PEG) ester.

The present invention further provides for the use of a composition comprising:
a) a cannabinoid; and
b) a polyethylene glycol (PEG) ester
   for the treatment, amelioration or prevention of a condition responsive to administration of a cannabinoid, wherein the prophylactically or therapeutically effective amount of the pharmaceutical composition comprising a polyethylene glycol (PEG) ester is from 0.08 to 1.25 times the prophylactically or therapeutically effective amount of an oral pharmaceutical composition which comprises the same amount of cannabinoid but does not comprise a polyethylene glycol (PEG).

The present invention provides a kit for the treatment, amelioration or prevention of a condition responsive to administration of a cannabinoid, said kit comprising:
a) a pharmaceutical composition comprising:
   i. a cannabinoid; and
   ii. a polyethylene glycol (PEG) ester
b) instructions for use.

The present invention provides a kit for the treatment, amelioration or prevention of a condition responsive to administration of a cannabinoid, said kit comprising:
a) a pharmaceutical composition comprising:
   i. a cannabinoid; and
   ii. a polyethylene glycol (PEG) ester
b) instructions for use
   wherein the bioavailability of the pharmaceutical composition comprising a polyethylene glycol (PEG) ester is from 0.8 to 12 times higher than the bioavailability of an oral pharmaceutical composition which comprises the same amount of cannabinoid but does not comprise a polyethylene glycol (PEG) ester.

The present invention further provides a kit for the treatment, amelioration or prevention of a condition responsive to administration of a cannabinoid, said kit comprising:
a) a pharmaceutical composition comprising:
   i. a cannabinoid; and
   ii. a polyethylene glycol (PEG) ester
b) instructions for use
   wherein the prophylactically or therapeutically effective amount of the pharmaceutical composition comprising a polyethylene glycol (PEG) ester is from 0.08 to 1.25 times the prophylactically or therapeutically effective amount of an oral pharmaceutical composition which comprises the same amount of cannabinoid but does not comprise a polyethylene glycol (PEG) ester.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 is a graph of the pharmacokinetics of oral administration of cannabidiol in different formulations in dogs.

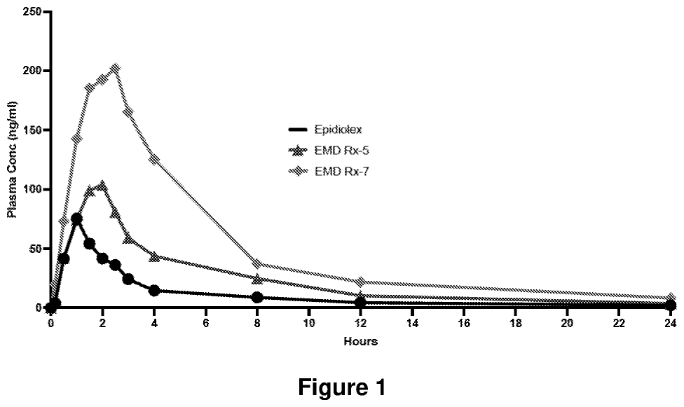

FIG. 2 is a graph of the average total CBD plasma exposures over 24 hours provided by EMD-RX5 compared to several commercially available CBD products with published data.

FIG. 3 is a box-plot graph comparing the median, 95% confidence intervals and total spread of CBD plasma concentrations recorded at specific time points over 24 provided by EMD-Rx5 and Epidiolex™.

FIG. 4 is a graph of the plasma concentration of CBD after dosing with EMD-Rx5 and Epidiolex™.

FIG. 5 is a graph of the plasma concentration of the major CBD metabolite 7-COOH—CBD after dosing with EMD-Rx5 and Epidiolex™.

FIG. 6 is a graph of the plasma concentration of the major CBD metabolite 7-OH—CBD after dosing with EMD-Rx5 and Epidiolex™.

FIG. 7 is a Table of exemplary formulations of CBD in capsules.

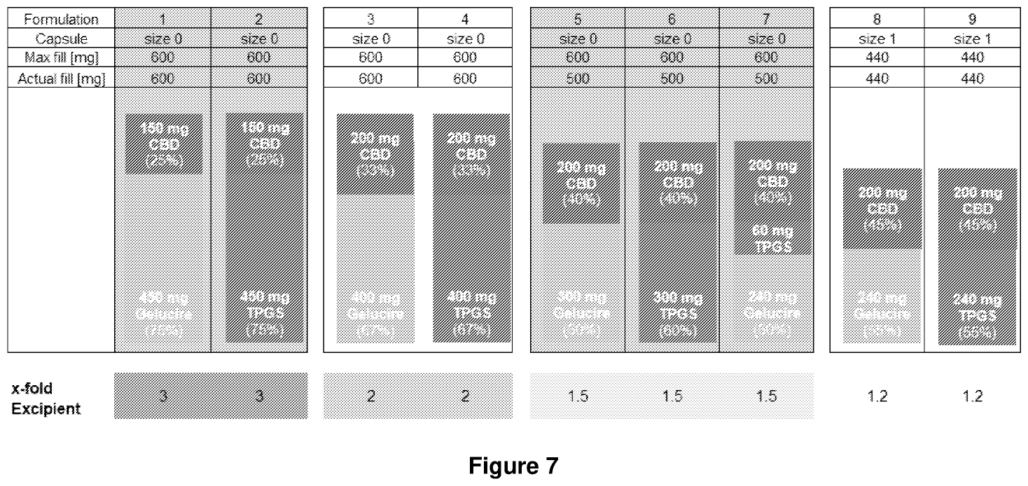

### DESCRIPTION OF INVENTION

### Detailed Description of the Invention

### Composition

Cannabinoids have a low oral bioavailability due a range of factors, including high lipophilicity, variable absorption, degradation of drug in the stomach; and significant first pass metabolism to active 11-OH-THC and inactive metabolites in the liver.

Cannabinoids are highly lipophilic and have poor water solubility, meaning that a large fraction of the dose may be lost in the gut and not absorbed. Even if the cannabinoid is mixed with an oil (for example in a capsule), it may precipitate out of solution from the oil in the gut due to the presence of water, thus lowering the absorption.

The present inventors have surprisingly found that delivery of a dose of cannabidiol in the presence of a PEG ester may provide the same treatment or prevention of symptoms of the condition that a higher dose of cannabidiol in the form of an oil provides. Thus, a lower dose of cannabidiol provided in the presence of a PEG ester may provide the same relief of symptoms of a range of diseases and conditions that are responsive to the oral administration of cannabinoids as a higher dose of cannabidiol provided in the form of an oil.

The present invention has found certain excipients that assist in increasing the bioavailability of oral cannabinoid dosage forms.

The present invention provides a pharmaceutical composition comprising:
a) a cannabinoid; and
b) a polyethylene glycol (PEG) ester.

Preferably the pharmaceutical composition of the present invention is oil-free, or contains only a minimal amount of an oil or lipid. For example, the composition of the present invention may contain a minimal amount of an oil or lipid that is less than 10%, less than 5%, less than 1%, less than 0.5% or less than 0.1% oil or lipid. The presence of an oil or lipid in a composition comprising cannabinoids may lead to adverse effect such as more rapid oxidation and lower bioavailability.

The composition may or may not contain water. Preferably, the composition does not contain water, i.e. it is non-aqueous. As cannabinoids are lipophilic and are not highly stable (i.e. are prone to degradation), the presence of water may lead to more repaid degradation of the active.

The present invention provides a pharmaceutical composition comprising:
a) a cannabinoid; and
b) a polyethylene glycol (PEG) ester
   wherein the bioavailability of the pharmaceutical composition comprising a polyethylene glycol (PEG) ester is from 0.8 to 12 times higher than the bioavailability of an oral pharmaceutical composition which comprises the same amount of cannabinoid but does not comprise a polyethylene glycol (PEG) ester.

The present invention provides a pharmaceutical composition comprising:
a) a cannabinoid; and
b) a polyethylene glycol (PEG) ester
   wherein the prophylactically or therapeutically effective amount of the pharmaceutical composition comprising a polyethylene glycol (PEG) ester is from 0.08 to 1.25 times the prophylactically or therapeutically effective amount of an oral pharmaceutical composition which comprises the same amount of cannabinoid but does not comprise a polyethylene glycol (PEG) ester.

The pharmaceutical composition which comprises the same amount of cannabinoid as the composition of the present invention, but which does not comprise a PEG ester, may also be termed the comparator composition in the present application.

Preferably the pharmaceutical composition of the present invention is delivered via the oral, rectal or vaginal route. Most preferably, the pharmaceutical composition of the present invention is delivered via the oral route. Therefore, the pharmaceutical composition of the present invention is most preferably an oral pharmaceutical composition. However, pharmaceutical compositions delivered to the vagina or rectum as capsules are contemplated.

Preferably the comparator oral pharmaceutical composition which does not comprise a polyethylene glycol (PEG) ester comprises less than 1% PEG ester, or less than 0.5%, 0.1%, or 0.05% PEG ester. Most preferably, the comparator composition comprises no detectable PEG ester.

It is common for cannabinoid compositions, particularly oral cannabinoid compositions, to include an oil as cannabinoids are highly lipophilic. The pharmaceutical composition of the present invention which does not comprise a polyethylene glycol (PEG) ester may comprise an oil. The oil may be, for example, sesame seed oil, bran oil, coconut oil, palm kernel oil or olive oil. Alternatively, the oral pharmaceutical composition of the present invention may be oil- or lipid-free, or contain only a minimal amount of an oil or lipid.

The oral pharmaceutical composition which does not comprise a polyethylene glycol (PEG) ester may further comprise an alcohol, such as a dehydrated alcohol. The alcohol may be, for example, ethanol. Alternatively, the composition may be alcohol free.

An example of an oral pharmaceutical composition which does not comprise a polyethylene glycol (PEG) ester and that may be used as the comparator composition is Epidiolex™ (also known as Epidyolex™). Each ml of Epidiolex™ contains 100 mg cannabidiol, 736 mg refined sesame oil, 79 mg of ethanol (equivalent to 10% v/v anhydrous ethanol), and 0.0003 mg benzyl alcohol. The activity of the present invention may be compared to Epidiolex™ to establish pharmacokinetics and the prophylactically or therapeutically effective dose or cannabinoid.

### Pharmacokinetics

The bioavailability of the composition of the present invention (and the comparator composition), may be measured by one or more of the following parameters:
AUC₀₋ₜ: The area under the plasma concentration vs. time curve, from time zero to time t.
AUC_{0-inf}: The area under the plasma concentration vs. time curve, from time zero to time infinity.
Tₘₐₓ: Time to the maximum measured plasma concentration.
Cₘₐₓ: Maximum measured plasma concentration over the time span specified.
t_{1/2}: Final time taken for the plasma concentration to be reduced by half.
AUCₗₐₛₜ: The area under the plasma concentration vs. time curve, from time zero to the last measurable concentration.

More preferably the bioavailability is measured by AUC₀₋ₜ.

Prior studies have determined that Cₘₐₓ and AUC following oral administration appears to be dose dependent. A dose of 10 mg CBD resulted in mean Cₘₐₓ of 2.47 ng/mL at 1.27 h, and a dose of 400 or 800 mg (co-administered with i.v. fentanyl) resulted in a mean Cₘₐₓ of 181 ng/mL (at 3.0 h) and 114 ng/mL (at 1.5 h) for 400 mg, and 221 ng/mL (at 3.0 h) and 157 ng/mL (at 4.0 h) for 800 mg, in two sessions, respectively (Guy and Robson, 2004b; Manini et al., 2015). A dose of 800 mg oral CBD in a study involving eight subjects reported a mean Cₘₐₓ of 77.9 ng/mL and mean Tₘₐₓ of 3.0 h (Haney et al., 2016). Although an increase in dose corresponds with an increase in Cₘₐₓ, the Cₘₐₓ between the higher doses of CBD does not greatly differ, suggesting a saturation effect (e.g., between 400 and 800 mg).

One hour after oral capsule administration containing 5.4 mg CBD, mean Cₘₐₓ was reported as 0.93 ng/mL (Nadulski et al., 2005a). A subset (n=12) consumed a standard breakfast meal 1 h after the capsules, which slightly increased mean Cₘₐₓ to 1.13 ng/mL. CBD remained detectable for 3-4 h after administration (Nadulski et al., 2005b).

The present invention provides a cannabinoid composition that:
has lower inter-participant dose variation;
has a more sustained release profile;
is able to maintain higher dose concentrations in later hours after administration; and/or
is safe and well tolerated
   compared to oral pharmaceutical cannabinoid compositions which do not comprise a polyethylene glycol (PEG) ester, particularly oral pharmaceutical cannabinoid compositions which contain an oil.

The terms “bioequivalence” or “bioequivalent” as used herein refer to a drug product or dosage form that has highly similar release and systemic absorption as compared to a reference drug. The U.S. Food, Drug and Cosmetic Act (21 U.S.C. § 505(j)(8)(B)(i)) provides that a drug is bioequivalent to a reference listed drug (RLD) if: “the rate and extent of absorption of the drug do not show a significant difference from the rate and extent of absorption of the listed drug when administered at the same molar dose of the therapeutic ingredient under similar experimental conditions in either a single dose or multiple doses”.

The phrase “enhanced bioavailability” as used herein refers to the increased proportion of an active pharmaceutical ingredient that enters the systemic circulation when introduced into the body as compared to a reference active pharmaceutical's bioavailability. Bioavailability can be determined by comparing the rate and extent of absorption of a test drug with a reference drug when administered at the same molar dose of the active therapeutic ingredient under similar experimental conditions in either a single dose or multiple doses. Typical pharmacokinetic parameters can be used to demonstrate enhanced bioavailability compared to the reference.

Preferably the bioavailability of a cannabinoid in a composition of the present invention is from 0.8 to 12 times higher than the bioavailability of an oral pharmaceutical composition which comprises the same amount of cannabinoid but does not comprise a polyethylene glycol (PEG) ester. For example, if the PEG ester is a stearoyl or lauroyl macrogol-32 glyceride, preferably the bioavailability of a cannabinoid in a composition of the present invention is from 1.2 to 3 times higher than the bioavailability of an oral pharmaceutical composition which comprises the same amount of cannabinoid but does not comprise a polyethylene glycol (PEG) ester. In another example, if the PEG ester is a vitamin E polyethylene glycol succinate, preferably the bioavailability of a cannabinoid in a composition of the present invention is from 3 to 5 times higher than the bioavailability of an oral pharmaceutical composition which comprises the same amount of cannabinoid but does not comprise a polyethylene glycol (PEG) ester.

The bioavailability of a cannabinoid in a composition of the present invention may be from 0.8 to 12 times higher than the bioavailability of an oral pharmaceutical composition which comprises the same amount of cannabinoid but does not comprise a polyethylene glycol (PEG) ester, for example 0.8, 1, 1.5, 2, 2.5, 3, 3.5, 4, 4.5, 5, 5.5, 6, 6.5, 7, 7.5, 8, 8.5, 9, 9.5, 10, 10.5, 11, 11.5 or 12 times higher. The bioavailability may be from 0.8 to 12, 1 to 11, 2 to 10, 2 to 5, or 1 to 5 times higher.

The prophylactically or therapeutically effective amount of the pharmaceutical composition comprising a polyethylene glycol (PEG) ester may be from 0.08 to 1.25 times the prophylactically or therapeutically effective amount of an oral pharmaceutical composition which comprises the same amount of cannabinoid but does not comprise a polyethylene glycol (PEG) ester, for example 0.08, 0.1, 0.12, 0.14, 0.16, 0.18, 0.2, 0.22, 0.24, 0.26, 0.28, 0.3, 0.32, 0.34, 0.36, 0.38, 0.4, 0.42, 0.44, 0.46, 0.48, 0.5, 0.52, 0.54, 0.56, 0.58, 0.6, 0.62, 0.64, 0.66, 0.68, 0.7, 0.72, 0.74, 0.76, 0.78, 0.8, 0.82, 0.84, 0.86, 0.88, 0.9, 0.92, 0.94, 0.96, 0.98, 1, 1.02, 1.04, 1.06, 1.08, 1.1, 1.12, 1.14, 1.16, 1.18, 1.2, 1.22 or 1.24 times less. The prophylactically or therapeutically effective amount may be from 0.08 to 1.2, 0.1 to 1.1, 0.2 to 1.0, 0.2 to 0.5, or 0.1 to 0.5 times less.

Preferably the AUC₀₋₂₄ of a cannabinoid in a composition of the present invention is from 0.8 to 12 times higher compared to the AUC₀₋₂₄ of an oral cannabinoid in a composition that does not comprise a PEG ester, more preferably 1.2 to 5 times higher compared to the AUC₀₋₂₄ of a cannabinoid in an oral composition that does not comprise a PEG ester.

Preferably the AUC₀₋₂₄ of a cannabinoid in a composition of the present invention is from 250 hr*ng/mL to 2000 hr*ng/mL.

Preferably the AUC₀₋₂₄ of a cannabinoid in a composition of the present invention is from 197.9% to 795.3% of a cannabinoid in a composition that does not comprise a PEG ester when the same dose of cannabinoid is provided in the present composition and the comparator composition.

Preferably the AUC_{0-inf} of a cannabinoid in a composition of the present invention is from 0.8 to 12 times higher compared to the AUC_{0-inf} of an oral cannabinoid in a composition that does not comprise a PEG ester, more preferably 1.2 to 5 times higher compared to the AUC_{0-inf} of a cannabinoid in an oral composition that does not comprise a PEG ester.

Preferably the AUC_{0-inf} of a cannabinoid in a composition of the present invention is from 250 hr*ng/mL to 2000 hr*ng/mL.

Preferably the AUC_{0-inf} of a cannabinoid in a composition of the present invention is from 208.0% to 806.2% of a cannabinoid in a composition that does not comprise a PEG ester when the same dose of cannabinoid is provided in the present composition and the comparator composition.

Preferably the Tₘₐₓ of a cannabinoid in a composition of the present invention reaches the same Tₘₐₓ as a cannabinoid in a comparator oral composition that does not comprise a PEG ester, such that t max is achieved in a time of from 0.5 to 2 times that of the comparator composition. Thus the compositions of the present invention may take from half as long to twice as long to reach Tₘₐₓ as comparator compositions when the same dose of cannabinoid is provided in the present composition and the comparator composition.

In one aspect, the Tₘₐₓ of a cannabinoid in a composition of the present invention may be greater than the comparator composition, but provides an equivalent dose exposure earlier than a cannabinoid in a composition that does not comprise a PEG ester. Thus, the cannabinoid in a composition of the present invention reaches the maximum concentration (Tₘₐₓ) of the comparator composition more rapidly than the comparator composition, and then keeps rising. The composition of the present invention may result in a greater final Tₘₐₓ but will reach the Tₘₐₓ of the comparator earlier than the comparator would.

Preferably the Tₘₐₓ of a cannabinoid in a composition of the present invention is from 1.2 h to 8 h, more preferably 2 h to 6 h, for example 3 h.

Preferably the Cₘₐₓ of a cannabinoid in a composition of the present invention is from 0.8 to 12 times higher compared to the Cₘₐₓ of a cannabinoid in an oral composition that does not comprise a PEG ester when the same dose of cannabinoid is provided in the present composition and the comparator composition, more preferably 1.2 to 5 times higher compared to the Cₘₐₓ of a cannabinoid in an oral composition that does not comprise a PEG ester.

Preferably the Cₘₐₓ of a cannabinoid in a composition of the present invention when administered orally to a human subject is from 40 ng/mL to 400 ng/mL, measured after administration of a dose of from 1 mg/kg/day to 25 mg/kg/day of a cannabinoid.

Preferably the Cₘₐₓ of a cannabinoid in a composition of the present invention when administered orally to a human subject is from 34.0% to 247.3% of a cannabinoid in a composition that does not comprise a PEG ester when the same dose of cannabinoid is provided in the present composition and the comparator composition.

Preferably the AUCₗₐₛₜ of a cannabinoid in a composition of the present invention is from 0.8 to 12 times higher compared to the AUCₗₐₛₜ of a cannabinoid in an oral composition that does not comprise a PEG ester when the same dose of cannabinoid is provided in the present composition and the comparator composition, more preferably 1.2 to 5 times higher compared to the AUCₗₐₛₜ of a cannabinoid in an oral composition that does not comprise a PEG ester.

Preferably the AUCₗₐₛₜ of a cannabinoid in a composition of the present invention when administered orally to a human subject is from 250 hr*ng/mL to 2,000 hr*ng/mL, measured after administration of a dose of from 1 mg/kg/day to 25 mg/kg/day of a cannabinoid.

Preferably the AUCₗₐₛₜ of a cannabinoid in a composition of the present invention when administered orally to a human subject is from 197.9% to 795.3% of a cannabinoid in a composition that does not comprise a PEG ester when the same dose of cannabinoid is provided in the present composition and the comparator composition.

Preferably the plasma concentration curve generated by the composition of the present invention has different characteristics from that generated by administration of a cannabinoid in an oral composition that does not comprise a PEG ester. For example, the composition of the present invention may arrive at Cₘₐₓ slower, and/or reduce from the Cₘₐₓ slower.

The present invention therefore provides a pharmaceutical composition comprising a cannabinoid and a polyethylene glycol (PEG) ester wherein the composition exhibits any one or more of:
a plasma concentration curve that increases at less than 55 ng/mL per hour up until the median Tₘₐₓ;
a plasma concentration curve that increases at a rate of from 30 to 55 ng/mL per hour up until the median Tₘₐₓ;
a plasma concentration curve that decreases at a rate of more than 5.5 ng/mL per hour after the median Tₘₐₓ;
a plasma concentration curve that decreases at a rate of from 6 to 4 ng/mL per hour after the median Tₘₐₓ
   when administered orally to a human subject, measured after administration of a dose of from 1 mg/kg/day to 25 mg/kg/day of a cannabinoid.

The present invention further provides a pharmaceutical composition comprising a cannabinoid and a polyethylene glycol (PEG) ester wherein the composition exhibits any one or more of:
a percentage of total drug delivered in 24 hours of from 10% to 30%, as measured between initial dose and T_{max(median)}. This is calculated as the AUC_{(0-Tmax(median)}/AUC₍₀₋ₗₐₛₜ₎, as measured by the total Area Under the Curve (AUC) plasma concentration;
a percentage of total drug delivered in 24 hours of 70% to 90%, as measured between T_{max(median)} and Tₘₐₓ₍ₗₐₛₜ₎. This is calculated as the AUC_{(Tmax(median)-Tmax(last)}/AUC₍₀₋ₗₐₛₜ₎
   when administered orally to a human subject, measured after administration of a dose of from 1 mg/kg/day to 25 mg/kg/day of a cannabinoid.

Preferably the dose administered per day is less than 20 mg/kg/day. More preferably the dose is less than 10 mg/kg/day, less than 5 mg/kg/day less than 2 mg/kg/day or less than 1 mg/kg/day. Most preferably, the dose administered per day is less than 10 mg/kg/day.

Preferably the plasma concentration curve generated by the composition of the present invention has a lower range of variability compared to oral cannabinoid composition that do not contain PEG ester, for example compositions that contain oils.

The present invention therefore provides a pharmaceutical composition comprising a cannabinoid and a polyethylene glycol (PEG) ester wherein the composition exhibits any one or more of:
a plasma concentration curve wherein the area under the curve of the plasma concentration has a standard deviation of from 20 to 126;
a plasma concentration curve wherein the area under the curve of the plasma concentration has a standard deviation of from 8 to 162 between dosing and 3 hours after dosing;
a plasma concentration curve wherein the area under the curve of the plasma concentration has a standard deviation of from 10 to 135 between 3 hours after dosing and 6 hours after dosing;
a plasma concentration curve wherein the area under the curve of the plasma concentration has a standard deviation of from 1 to 51 between 6 hours after dosing and 12 hours after dosing
   when administered orally to a human subject, measured after administration of a dose of from 1 mg/kg/day to 25 mg/kg/day of a cannabinoid.

The present invention further provides a pharmaceutical composition comprising a cannabinoid and a polyethylene glycol (PEG) ester wherein the composition exhibits any one or more of:
a plasma concentration curve wherein the area under the curve of the plasma concentration has a standard deviation of from 22 to 163 between dosing and Cₘₐₓ;
a plasma concentration curve wherein the area under the curve of the plasma concentration has a standard deviation of from 16 to 135 between Cₘₐₓ and 6 h after dosing;
a plasma concentration curve wherein the area under the curve of the plasma concentration has a standard deviation of from 17 to 139 between Cₘₐₓ and 12 h after dosing
   when administered orally to a human subject, measured after administration of a dose of from 1 mg/kg/day to 25 mg/kg/day of a cannabinoid.

When compared to a comparator composition, for example Epidiolex™, the cannabinoid composition of the present invention exhibits any one or more of:
a corresponding interval AUC for the cannabinoid composition of the present invention that is substantially larger than Epidyolex™ in absolute terms (492.2 hr*ng/mL versus 306.0 hr*ng/mL)
a corresponding interval AUC for the cannabinoid composition of the present invention that is substantially larger than Epidyolex™ in proportion (to total AUC) terms (82.5% versus 49.0%);
a rate of change of the concentration cannabinoid in plasma that is similar at each time point for the cannabinoid composition of the present invention compared to the comparator composition (19.4 ng/mL/hr versus 17.4 ng/mL/hr);
a plasma concentration curve wherein the concentration of cannabinoid in plasma is substantially larger at each time point for the cannabinoid composition of the present invention compared to the comparator composition;
a plasma concentration curve wherein for Time=4, 5, and 6, the average concentration for the cannabinoid composition of the present invention is more than double that of Epidyolex™ (84.7 ng/mL versus 38.2 ng/ML; 65.9 ng/mL versus 27.4 ng/mL; 50.2 ng/mL versus 22.0 ng/mL), with the latter two time points being statistically meaningful at least at the 5 percent level of statistical significance (p=0.035; p=0.047)
   when administered orally to a human subject, measured after administration of a dose of from 1 mg/kg/day to 25 mg/kg/day of a cannabinoid when the same dose of cannabinoid is provided in the present composition and the comparator composition.

Preferably the cannabinoid in a composition of the present invention generates a different metabolite profile in plasma to that generated by administration of a cannabinoid in an oral composition that does not comprise a PEG ester when the same dose of cannabinoid is provided in the present composition and the comparator composition. For example, the cannabinoid in a composition of the present invention may generate a lower concentration in plasma of 7-COOH—CBD and/or 7-OH—CBD as it is metabolised than the concentration in plasma of 7-COOH—CBD and/or 7-OH—CBD generated by metabolism of a cannabinoid in an oral composition that does not comprise a PEG ester.

The concentration in plasma of 7-COOH—CBD produced by metabolism of CBD delivered in in a composition of the present invention may be at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24% or 25% lower than the concentration in plasma of 7-COOH—CBD produced by metabolism of CBD delivered in an oral composition that does not comprise a PEG ester when the same dose of cannabinoid is provided in the present composition and the comparator composition.

The concentration in plasma of 7-OH—CBD produced by metabolism of CBD delivered in in a composition of the present invention may be at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24% or 25% lower than the concentration in plasma of 7-OH—CBD produced by metabolism of CBD delivered in an oral composition that does not comprise a PEG ester when the same dose of cannabinoid is provided in the present composition and the comparator composition.

Preferably the pharmaceutical composition is for oral delivery. The pharmaceutical composition allows administration of a cannabinoid to the gastrointestinal (GI) tract of the subject. Preferably, the cannabidiol enters the blood stream via absorption in the GI tract and is systemically available to the subject. However, vaginal or rectal delivery is also contemplated. Vaginal or rectal delivery will still result in blood stream via absorption and systemic delivery.

Preferably the pharmaceutical composition is held within a soft capsule, also known as a liquid capsule. For example, the soft capsule may be a gelatine soft capsule. Alternatively, the soft capsule may be a vegetarian soft capsule, such as those made from tapioca or modified starch. An example of vegetarian soft capsules is Versagel™ capsules.

Alternatively, the pharmaceutical composition may be held within a hard capsule, also known as a liquid-filled hard capsule. The hard capsule may be a gelatine hard capsule or a collagen hard capsule. The hard capsule may be a vegetarian hard capsule, such as those made from hydroxypropyl methylcellulose (aka hypromellose or HPMC), or pullulan.

Preferably the pharmaceutical composition is held within a capsule of size 2 to size 00. The capsule size may be determined based on the amount of cannabinoid desired to be held in each capsule. Preferably the capsule of size 2 to size 00 is a soft capsule. Alternatively, the capsule of size 2 to size 00 may be a hard capsule.

### Polyethylene Glycol Ester

Preferably the polyethylene glycol (PEG) ester is amphiphilic and possesses both hydrophilic and lipophilic functional groups.

Preferably the PEG ester is miscible with water. Preferably the PEG ester is a water dispersible surfactant. Preferably the PEG ester is a non-ionic surfactant.

Preferably the PEG ester is miscible with cannabinoids. More preferably the PEG ester is miscible with highly lipophilic cannabinoids such as cannabidiol and THC.

The advantage of a PEG ester that is miscible with both water and lipophilic cannabinoids is that the PEG ester forms a “bridge” between the water and the cannabinoid, such that the cannabinoid remains in solution once the cannabinoid is in the aqueous gut environment. This reduces the likelihood of the cannabinoid precipitating out of the dosage form and forming lipophilic oily droplets in the gut. Such lipophilic oily droplets are poorly absorbed by the gut, and therefore keeping the cannabinoid in a soluble form by the use of the PEG ester increases the absorbability and thus bioavailability of the cannabinoid.

Preferably the PEG ester has an HLB value of from 8 to 16. The “HLB” refers to a value that is used to index and describe a surfactant according to its relative hydrophobicity/hydrophilicity, relative to other surfactants. HLB number of a surfactant is defined as HLB=20*MH/MT, where MH and MT are the mass of the hydrophilic head group and the total surfactant mass, respectively. The HLB is measured on a scale of 0 to 20, with an HLB value of 0 corresponding to a completely lipophilic/hydrophobic molecule, and a value of 20 corresponding to a completely hydrophilic/lipophobic molecule. A surfactant's HLB value is an indication of the molecular balance of the hydrophobic and hydrophilic portions of the surfactant, which is an amphipathic molecule. More preferably the PEG ester has an HLB value of from 9 to 15, more preferably from 12 to 14. GELUCIRE™ 50/13 has an HLB of 13, GELUCIRE™ 44/14 has an HLB of 13 and Kolliphor™ TPGS has an HLB of 13.

The polyethylene glycol (PEG) ester may be chosen from the list comprising: stearoyl or lauroyl macrogol-32 (also known as polyoxyl-32) glycerides formed from hydrogenated palm oil PEG-32 esters (for example Gelucires such as GELUCIRE™ 50/13 a stearoyl polyoxyl-32 glyceride or GELUCIRE™ 44/14 a lauroyl polyoxyl-32 glyceride); vitamin E polyethylene glycol succinates formed from esterification of vitamin E succinate with polyethylene glycol (for example vitamin E TPGS such as Kolliphor™ TPGS); lauroyl PEG-32 glycerides; or PEG-32 esters of palmitic and stearic acid, and combinations thereof. Preferably the polyethylene glycol (PEG) ester is stearoyl or lauroyl macrogol-32 glyceride formed from hydrogenated palm oil PEG-32 esters or vitamin E polyethylene glycol succinates formed from esterification of vitamin E succinate with polyethylene glycol. Some PEG ester products, such as some GELUCIRE™ products, may contain oils or lipids (triglycerides). Preferably the PEG ester used in the compositions of the present invention are those PEG ester products that do not contain an oil or lipid, or that contain a minimal amount of an oil or lipid. For example, “low HLB Gelucires” (e.g. GELUCIRE™ 43/01), contain 100% triglycerides while the “high HLB Gelucires” (e.g. GELUCIRE™ 50/13 or GELUCIRE™ 44/14) contain only minimal amounts of triglycerides. GELUCIRE™ 44/14 and GELUCIRE™ 50/13 are both saturated polyglycolized glycerides.

The polyethylene glycol (PEG) ester may be a mixture of PEG esters. For example, the PEG ester may be a mixture of stearoyl or lauroyl macrogol-32 glyceride and vitamin E polyethylene glycol succinate.

The ratio of the PEG esters may be from 5:95 to 95:5 or each PEG ester. If more than two PEG esters are used, the ratios may be adjusted as required. For example, if the mixture of PEG esters is a mixture of stearoyl or lauroyl macrogol-32 glyceride and vitamin E polyethylene glycol succinate, the ratio of the two PEG esters may be selected from the following:

| Parts stearoyl or lauroyl | Parts vitamin E polyethylene |
|---|---|
| macrogol-32 glyceride | glycol succinate |
| 95 | 5 |
| 90 | 10 |
| 85 | 15 |
| 80 | 20 |
| 75 | 25 |
| 70 | 30 |
| 65 | 35 |
| 60 | 40 |

As shown above, preferably there is more stearoyl or lauroyl macrogol-32 glyceride than vitamin E polyethylene glycol succinate in any mixture of these PEG esters. As the bioavailability of cannabinoids appears to be higher when provided in vitamin E polyethylene glycol succinate than stearoyl or lauroyl macrogol-32 glyceride, the likelihood of dose dumping may increase with increasing amounts of vitamin E polyethylene glycol succinate. The bioavailability of cannabinoids in a composition of the present invention may be manipulated by varying the relative amounts of stearoyl or lauroyl macrogol-32 glyceride to vitamin E polyethylene glycol succinate. Such manipulation of the bioavailability of cannabinoids may be carried out with other combinations of PEG esters if one PEG ester provides higher bioavailability of a cannabinoid than another PEG ester.

Furthermore, vitamin E polyethylene glycol succinate is more expensive than stearoyl or lauroyl macrogol-32 glyceride and commercially it may be desirable to reduce or limit its presence in a composition.

Preferably the PEG ester is liquid at a temperature above room temperature (between 20° C.-25° C.). For example, the PEG ester may be liquid at 26° C., 27° C., 28° C., 29° C., 30° C., 31° C., 32° C., 33° C., 34° C., 35° C., 36° C., 37° C., 38° C., 39° C., or 40° C. or higher than 40° C. For example, the PEG ester may melt at from 37° C. to 55° C. Thus, the melting point of the PEG ester is preferably above room temperature. This means that when the cannabinoid is mixed with the PEG ester, the PEG ester can be a liquid at a temperature that will allow suitable dissolution of the cannabinoid into the PEG and convenient filling of capsules.

The presence of a PEG ester that is liquid above room temperature and a gel or solid at room temperature assist in the manufacture, storage and distribution of the pharmaceutical composition comprising a cannabinoid. The liquid/gel profile of the pharmaceutical composition allows a dosage form, such as a hard or soft capsule, to be easily filled at a temperature above room temperature, as the pharmaceutical composition is liquid. The liquid is then allowed to cool to room temperature, for example in the hard or soft capsule, forming a dosage form. As the pharmaceutical composition is a gel or solid at room temperature, for example within a dosage form such as a hard or soft capsule, the pharmaceutical composition will not leak from the dosage form.

Preferably the pharmaceutical composition comprises from 100 mg/capsule to 500 mg/capsule of PEG ester. More preferably the pharmaceutical composition comprises from 150 mg/capsule to 400 mg/capsule of PEG ester, or from 200 mg/capsule to 300 mg/capsule. The pharmaceutical composition may comprise 100, 150, 200, 250, 300, 350, 400, 450 or 500 mg/capsule of PEG ester. Preferably the pharmaceutical composition comprises 250 mg/capsule of PEG ester.

For example, the pharmaceutical composition may comprise at least 50 mg cannabinoid/250 mg PEG ester per single capsule of size 2, but amounts up to 150 mg cannabinoid/400 mg PEG ester may be incorporated into a size 0 capsule. The skilled person can easily calculate the relative amounts of cannabinoid/PEG ester that are suitable for the chosen capsule size.

Preferably the pharmaceutical composition comprises a ratio of 1:10 w/w cannabinoid:PEG ester, or a 1:9, 1:8, 1:7, 1:6, 1:5, 1:4, 1:3, 1:2 or 1:1 w/w ratio of cannabinoid:PEG ester. The ratio of cannabinoid:PEG ester may be 2:10, 2:9, 2:8, 2:7, 2:6, 2:5, 2:4 or 2:3 w/w cannabinoid:PEG ester. Preferably the ratio of cannabinoid:PEG ester is from 1:5 to 1:1.5 cannabinoid:PEG ester.

Without being held to any theory, it seems that the ratio of cannabinoid to PEG ester is important in keeping the cannabinoid in solution. If the ratio is adjusted to include too much cannabinoid, then the active may crystalise out of the PEG ester solution. If the ratio is adjusted to include too much PEG ester, then there is too little cannabinoid to conveniently dose a subject as either the capsules are too big to easily swallow or the subject needs to take a large number of capsules to make the dose up to the required daily dose.

Some examples of cannabinoid to excipient ratios are provided in Table 1. The shaded cells represent examples of preferable ratios. Preferably the ratios are from about 10% to 25%, more preferably from 15% to 20%.

TABLE 1
Examples of cannabinoid to excipient ratios

| | Capsule size and max. capacity [mg] for powder density of ~ 0.8 g/ml | | | | |
|---|---|---|---|---|---|
| mg CBD | 300 | 400 | 600 | 620 | 760 |
| per capsule | size 2 | size 1 | size 0 | size 0el | size 00 |
| 50 | 16.7% | 12.5% | 8.3% | 8.1% | 6.6% |
| 100 | 33.3% | 25.0% | 16.7% | 16.1% | 13.2% |
| 150 | 50.0% | 37.5% | 25.0% | 24.2% | 19.7% |
| 200 | 66.7% | 50.0% | 33.3% | 32.3% | 26.3% |
| 250 | 83.3% | 62.5% | 41.7% | 40.3% | 32.9% |
| 300 | 100.0% | 75.0% | 50.0% | 48.4% | 39.5% |
| 350 | | 87.5% | 58.3% | 56.5% | 46.1% |

It is further believed that the ratio of active cannabinoid to PEG ester provides additional advantages. The compositions of the present invention generate a different metabolite profile than comparator compositions that comprise a significant amount of oil but no PEG ester. The compositions of the present invention preferably generate less of the metabolite 7-COOH—CBD and the metabolite 7-OH—CBD. The compositions of the present invention also preferably generate later Cₘₐₓ peaks for these metabolites. This may be a result of less first pass metabolism of the cannabinoid, particularly less first pass metabolism of cannabidiol.

An advantage of a composition that produces later and lower metabolite production is that this indicates a reduced “dose dumping” effect. Dose dumping is a phenomenon in which relatively large amount of drug in a controlled release formulation is quickly released and a potentially toxic quantity of the drug is introduced into systemic circulation. The avoidance of dose dumping by providing the cannabinoid active in a composition with a PEG ester at a particular ratio allows reduced first pass metabolism, a slower rise to Cₘₐₓ, reduced metabolite production, and reduced GI effects.

### Cannabinoid

The term cannabinoid includes compounds which interact with the cannabinoid receptor and various cannabinoid mimetics, such as certain tetrahydropyran analogs (e.g., Δ⁹-tetrahydrocannabinol, Δ⁸-tetrahydro-cannabinol, 6,6,9-trimethyl-3-pentyl-6H-dibenzo[b,d]pyran-1-ol, 3-(1,1-dimethylheptyl)-6,6a,7,8,10,10a-hexahydro-1-hydroxy-6,6-dimethyl-9H-dibenzo[b,d]pyran-9-one, (−)-(3S,4S)-7-hydroxy-Δ6-tetrahydrocannabinol-1,1-dimethylheptyl, (+)-(3S,4S)-7-hydroxy-Δ6-tetrahydrocannabinol-1,1-dimethylheptyl, 11-hydroxy-Δ⁹-tetrahydrocannabinol, and Δ8-tetrahydrocannabinol-11-oic acid)); certain piperidine analogs (e.g., (−)-(6S,6aR,9R,10aR)-5,6,6a,7,8,9,10,10a-octahydro-6-methyl-3-[(R)-1-methyl-4-phenylbutoxy]-1,9-phenanthridinediol-1-acetate)); certain aminoalkylindole analogs (e.g., (R)-(+)-[2,3-dihydro-5-methyl-3-(-4-morpholinylmethyl)-pyrrolo[1,2,3-de]-1,4-benzoxazin-6-yl]-1-naphthalenyl-methanone); and certain open pyran ring analogs (e.g., 2-[3-methyl-6-(1-methylethenyl)-2-cyclohexen-1-yl]-5-pentyl-1,3-benzenediol and 4-(1,1-dimethylheptyl)-2,3′-dihydroxy-6′alpha-(3-hydroxypropyl)-1′,2′,3′,4′,5′,6′-hexahydrobiphenyl).

Preferably, the cannabinoid is chosen from the list comprising: cannabidiol, cannabinol, cannabigerol, cannabichromene, and Δ⁹-tetrahydrocannabinol (THC). Most preferably, the cannabinoid is cannabidiol (CBD).

Cannabidiol, as used herein, refers to 2-[3-methyl-6-(1-methylethenyl)-2-cyclohexen-1-yl]-5-pentyl-1,3-benzenediol. The synthesis of cannabidiol is described, for example, in Petilka et al., Helv. Chim. Acta, 52:1102 (1969) and in Mechoulam et al., J. Am. Chem. Soc., 87:3273 (1965), which are hereby incorporated by reference.

The term “cannabidiol” may include pre-cursors of cannabidiol such as cannabidiolic acid (CBDA or CBD-A). In one form of the invention the term “cannabidiol” only covers cannabidiol per se. In another form of the invention, the term “cannabidiol” covers both cannabidiol and precursor compounds. In another form of the invention, the term “cannabidiol” covers both cannabidiol and the precursor compound, cannabidiolic acid.

Preferably the pharmaceutical composition comprises from 25 mg to 750 mg total cannabinoid. The composition may comprise from 25 mg to 650 mg, 25 mg to 550 mg, 25 mg to 450 mg, 25 mg to 350 mg, 25 mg to 250 mg, 25 mg to 150 mg, 25 mg to 100 mg, or 25 mg to 50 mg cannabinoid. The composition may comprise from 25 mg to 750 mg, 50 mg to 750 mg, 100 mg to 750 mg, 150 mg to 750 mg, 250 mg to 750 mg, 350 mg to 750 mg, 450 mg to 750 mg, 550 mg to 750 mg, or 650 mg to 750 mg. Preferably the composition comprises 50 mg to 150 mg cannabinoid. The composition may be in a single dose form (for example a single capsule) or the composition may be in several dosage forms (eg several capsules).

The pharmaceutical composition may comprise 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg or 750 mg total cannabinoid. More preferably the composition comprises 50 mg, 100 mg or 150 mg total cannabinoid.

Preferably the pharmaceutical composition comprises from 25 mg to 200 mg cannabidiol. The composition may comprise from 25 mg to 650 mg, 25 mg to 550 mg, 25 mg to 450 mg, 25 mg to 350 mg, 25 mg to 250 mg, 25 mg to 150 mg, 25 mg to 100 mg, or 25 mg to 50 mg cannabidiol. The composition may comprise from 25 mg to 750 mg, 50 mg to 750 mg, 100 mg to 750 mg, 150 mg to 750 mg, 250 mg to 750 mg, 350 mg to 750 mg, 450 mg to 750 mg, 550 mg to 750 mg, or 650 mg to 750 mg cannabidiol. Preferably the composition comprises 50 mg to 150 mg cannabidiol. The composition may be in a single dose form (for example a single capsule) or the composition may be in several dosage forms (eg several capsules).

The pharmaceutical composition may comprise 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg or 750 mg cannabidiol. More preferably the composition comprises 50 mg, 100 mg or 150 mg cannabidiol.

The pharmaceutical composition may comprise from 15% w/w to 50% w/w cannabinoid. The composition may comprise from 15% w/w to 50% w/w cannabinoid, 20% w/w to 50% w/w, 25% w/w to 50% w/w, 30% w/w to 50% w/w, 35% w/w to 50% w/w, 40% w/w to 50% w/w or 45% w/w to 50% w/w cannabinoid. The composition may comprise from 15% w/w to 45% w/w cannabinoid, 15% w/w to 40% w/w, 15% w/w to 35% w/w, 15% w/w to 30% w/w, 15% w/w to 25% w/w, or 15% w/w to 20% w/w cannabinoid. The composition may comprise from 16% w/w to 35% w/w cannabinoid.

The total daily dose of cannabinoid administered to a subject per day is preferably from 50 mg/day to 4000 mg/day. More preferably the total daily dose is from 50 mg/day to 1500 mg/day, or from 50 mg/day to 1400 mg/day, from 100 mg/day to 1000 mg/day, from 100 mg/day to 600 mg/day, or from 150 mg/day to 600 mg/day.

The total daily dose of cannabinoid administered to a subject per day is preferably 50 mg/day, 100 mg/day, 150 mg/day, 200 mg/day, 250 mg/day, 300 mg/day, 350 mg/day, 400 mg/day, 450 mg/day, 500 mg/day, 550 mg/day, 600 mg/day, 650 mg/day, 700 mg/day, 750 mg/day, 800 mg/day, 850 mg/day, 900 mg/day, 950 mg/day, 1000 mg/day, 1050 mg/day, 1100 mg/day, 1150 mg/day, 1200 mg/day, 1250 mg/day, 1300 mg/day, 1350 mg/day, 1400 mg/day, 1450 mg/day, 1500 mg/day, 1550 mg/day, 1600 mg/day, 1650 mg/day, 1700 mg/day, 1750 mg/day, 1800 mg/day, 1850 mg/day, 1900 mg/day, 1950 mg/day, 2000 mg/day, 2050 mg/day, 2100 mg/day, 2150 mg/day, 2200 mg/day, 2250 mg/day, 2300 mg/day, 2350 mg/day, 2400 mg/day, 2450 mg/day, 2500 mg/day, 2550 mg/day, 2600 mg/day, 2650 mg/day, 2700 mg/day, 2750 mg/day, 2800 mg/day, 2850 mg/day, 2900 mg/day, 2950 mg/day, 3000 mg/day, 3050 mg/day, 3100 mg/day, 3150 mg/day, 3200 mg/day, 3250 mg/day, 3300 mg/day, 3350 mg/day, 3400 mg/day, 3450 mg/day, 3500 mg/day, 3550 mg/day, 3600 mg/day, 3650 mg/day, 3700 mg/day, 3750 mg/day, 3800 mg/day, 3850 mg/day, 3900 mg/day, 3950 mg/day or 4000 mg/day.

Preferably the total daily dose is about 150 mg/day, from 300 to 600 mg/day or from 1400 to 1500 mg/day.

The total daily dose of cannabinoid administered to a subject per day is preferably from 1 mg/kg/day to 25 mg/kg/day. The total daily dose may be from 1 mg/kg/day to 20 mg/kg/day, from 1 mg/kg/day to 15 mg/kg/day, from 1 mg/kg/day to 10 mg/kg/day, or from mg/kg/day to 5 mg/kg/day. The total daily dose may be from 5 mg/kg/day to 25 mg/kg/day, 10 mg/kg/day to 25 mg/kg/day, 15 mg mg/kg/day to 25 mg/kg/day, or 20 mg/kg/day to 25 mg/kg/day. The total daily dose may be 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5 or 25 mg/kg/day.

The dose of cannabinoid, such as cannabidiol, being administered to the subject may be increased from a lower dose to a higher dose over a time period. For example, the subject may be first administered a dose of 50 mg per day total cannabinoid. Over the course of several weeks, this dose may be increased to 60 mg, or 100 mg, or 150 mg. The increase is dosage may be carried out for a range of reasons, including a wish to avoid side-effects caused by initial administration of high doses of cannabinoid, such as cannabidiol, to a subject unused to such doses, and increasing the dose if the subject is not responding sufficiently to the initial lower dose.

The compositions may contain more than one cannabinoid. For example, the composition of the present invention may contain a combination of two, three or more cannabinoids.

Preferably the pharmaceutical composition comprises a majority of one cannabinoid. More preferably, the composition may comprise at least 80%, 81%, 82%, 83%, 84%, 85%, 86%, 87%, 88%, 89%, 90%, 91%, 92%, 93%, 94%, 95%, 96%, 97%, 98%, 99% of one cannabinoid. The remaining % of the composition may be other cannabinoids or other impurities. Preferably the majority cannabinoid is cannabidiol (CBD).

For example, the composition may comprise greater than or equal to 98% (w/w) CBD and less than or equal to 2% (w/w) other cannabinoids. The less than or equal to 2% (w/w) other cannabinoids may comprise the cannabinoids tetrahydrocannabinol (THC); cannabidivarin (CBDV); cannabinol (CBN); cannabigerol (CBG); tetrahydrocannabivarin; (THCV); cannabichromene (CBC); nantradol hydrochloride; nabilone and other naturally occurring cannabinoids.

Suitable pharmaceutically acceptable salts for which a salt of cannabinoids can be made include conventionally used non-toxic salts, for example a salt with an inorganic base such as an alkali metal salt (such as sodium salt and potassium salt), an alkaline earth metal salt (such as calcium salt and magnesium salt), an ammonium salt; or a salt with an organic base, for example, an amine salt (such as methylamine salt, dimethylamine salt, cyclohexylamine salt, benzylamine salt, piperidine salt, ethylenediamine salt, ethanolamine salt, diethanolamine salt, triethanolamine salt, tris(hydroxymethylamino) ethane salt, monomethyl-monoethanolamine salt, procaine salt and caffeine salt), a basic amino acid salt (such as arginine salt and lysine salt), tetraalkyl ammonium salt and the like, or other salt forms that enable the cannabinoid to remain soluble in a liquid medium, or to be prepared and/or effectively administered in a liquid medium, preferable an aqueous medium. The above salts may be prepared by a conventional process, for example from the corresponding acid and base or by salt interchange.

Examples of suitable pharmaceutically acceptable salts for which a salt of cannabinoids can be made include inorganic acid addition salts such as hydrochloride, hydrobromide, sulfate, phosphate, and nitrate; organic acid addition salts such as acetate, propionate, succinate, lactate, glycolate, malate, tartrate, citrate, maleate, fumarate, methansulfonate, p-toluenesulfonate, and ascorbate; salts with acidic amino acid such as aspartate and glutamate; alkali metal salts such as sodium salt and potassium salt; alkaline earth metal salts such as magnesium salt and calcium salt; ammonium salt; organic basic salts such as trimethylamine salt, triethylamine salt, pyridine salt, picoline salt, dicyclohexylamine salt, and N,N′-dibenzylethylenediamine salt; and salts with basic amino acid such as lysine salt and arginine salt. The salts may be in some cases hydrates or ethanol solvates.

In one embodiment at least a portion of at least one of the cannabinoids present in the composition is isolated from Cannabis plant material. Preferably at least a portion of the CBD present in the composition is isolated from Cannabis plant material.

In a further embodiment of the invention substantially all of at least one of the cannabinoids present in the composition is isolated from Cannabis plant material. Preferably substantially all of the CBD present in the composition is isolated from Cannabis plant material.

In a further embodiment of the invention substantially all of the cannabinoids present in the composition are isolated from Cannabis plant material.

Preferably the Cannabis plant material is from Cannabis sativa, Cannabis indica, or Cannabis ruderalis plants. Preferably the Cannabis plant is a high-CBD containing Cannabis chemotype.

In a further embodiment of the invention at least a portion of at least one of the cannabinoids present in the composition is prepared synthetically. Preferably at least a portion of the CBD present in the composition is prepared synthetically.

In a further embodiment of the invention substantially all of at least one of the cannabinoids present in the composition is prepared synthetically. Preferably substantially all of the CBD present in the composition is prepared synthetically.

Preferably substantially all of the cannabinoids present in the composition are prepared synthetically.

If at least a portion or substantially all of the CBD present in the composition is synthetic, then preferably the synthetic CBD only contains the (−) CBD enantiomer.

The composition may contain both cannabinoids isolated from Cannabis plant material and synthetically prepared cannabinoids. For example, the composition may contain synthetically prepared CBD and a mixture of other cannabinoids isolated from Cannabis plant material.

Preferably the composition comprises not more than 2.0% or 1.5% (w/w) THC based on total amount of cannabinoid in the preparation. More preferably the composition comprises about 0.01% to about 0.1% (w/w) THC based on total amount of cannabinoid in the preparation. More preferably the composition comprises about 0.02% to about 0.05% (w/w) THC based on total amount of cannabinoid in the preparation. More preferably the composition comprises 0% (w/w) THC.

### Antioxidant

Preferably the pharmaceutical composition comprises an antioxidant. The antioxidant may be selected from the list comprising butylated hydroxytoluene (BHT), butylated hydroxyanisole (BHA), ascorbyl palmitate, alpha-tocopherol, sodium metabisulfite, propyl gallate, cysteine, and combinations thereof. More preferably the antioxidant is BHT, BHA or combinations thereof.

Preferably the composition comprises not more than 1 mg/capsule antioxidant. Preferably the pharmaceutical composition comprises from 0.01 mg/capsule to 1 mg/capsule antioxidant, or from 0.05 mg/capsule to 1 mg/capsule, 0.5 mg/capsule to 1 mg/capsule antioxidant. For example, the composition may comprise 0.01, 0.05, 0.5 or 1 mg/capsule antioxidant.

### Stability

It is known that compositions comprising cannabinoids in oil lack stability and the active cannabinoids are subject to degradation due to temperature, humidity, and oxygen. The phrase “enhanced stability,” “stable,” or “enhanced oxidative stability” as used herein refers to the increased stability of a compound after isolation or synthesis, during manufacturing, or in its final dosage form. “Enhanced stability” refers to the ability of the compound to resist oxidation, reduction, or degradation and remain in a substantially chemically pure form. Most cannabinoids are highly susceptible to oxidation. Current formulations of cannabinoids require storage in light-proof containers and storage under refrigerated (2-8° C.) or cool (8-15° C.) conditions. The compositions and formulations described herein have enhanced stability compared to the current formulations because they are less susceptible to oxidation and do not require refrigeration for stability (although their stability can be further enhanced by cold storage).

Preferably the pharmaceutical composition of the present invention comprising a cannabinoid and a PEG ester is stable for at least 3 months, at least 4 months, at least 5 months, or at least 6 months. More preferably the composition of the present invention is stable for at least 12 months.

The term “chemically stable” or “chemical stability” of a composition of the present invention refers to the ability of the composition and/or cannabinoid(s) in the composition to resist change in its chemical properties over time. Chemical instability of a composition may be manifested by decrease in the amount of the cannabinoid, e.g., THC or CBD. Chemical degradation of THC may occur due to conversion of TCH to cannabinol (CBN). Chemical degradation of CBD may occur due to oxidation, resulting in monomeric and dimeric hydroxyquinones. Physical instability of an emulsion may be manifested in any of the following: flocculation, creaming, coalescence and Ostwald ripening. Determination whether an emulsion has lost its physical stability may be carried out in any of the following techniques: measurement of particle size, light scattering, focused beam reflectance measurement, centrifugation, rheology or a combination thereof. Stability may be affected by temperature, relative humidity (RH), light and other factors.

In one embodiment, the composition is stable at room temperature (21-25° C.), for a period of time that is at least about 12 months, for at least about 18 months, or for at least about 24 months, at 25° C.±2° C./40% RH±5% RH, with <20% decrease, <10% decrease, or preferably <5% decrease, in active ingredient content, e.g., in cannabinoid content (e.g., total, THC or CBD), and no change on dispersion in 37° C. water over the respective time period 12 months. It is also an object of the present invention to provide the composition as mentioned above, wherein the composition is stable at 5° C.±3° C./40% RH±5% RH for a period of time that is at least about 6 months, preferably for at least about 12 months, more preferably for at least about 18 months, more preferably for at least about 24 months, with <20% decrease, <10% decrease, or preferably <5% decrease, in cannabinoid content and no change on dispersion in 37° C. water over the relevant time frame. It is also an object of the present invention to provide the composition as mentioned above, wherein the composition is stable at about 40° C.±2° C./75% RH±5% RH for a period of time that is at least about 2 months, preferably for at least about 6 months, more preferably for at least about 9 months, even more preferably for at least about 12 months, and most preferably for at least about 24 months, with <20% decrease, <10% decrease, or preferably <5% decrease, in cannabinoid content and no change on dispersion in 37° C. water over the relevant respective time frame.

In a further embodiment, the composition is stable at room temperature (21-25° C.), for a period of time that is at least about 12 months, at 25° C.±2° C./40% RH±5% RH, with <20% decrease in cannabinoid content and no change on dispersion in 37° C. water over the period of time. In a further embodiment, the formulation is stable for at least about 18 months. In a further embodiment, the formulation is stable for at least about 24 months. In a further embodiment, there is <10% decrease in cannabinoid content, e.g., total, THC or CBD. In a further embodiment, there is <5% decrease in cannabinoid content.

In a further embodiment, the composition is stable at 5° C.±3° C./40% RH±5% RH for a period of time that is at least about 6 months, with <20% decrease in cannabinoid content, and no change on dispersion in 37° C. water over the period of time. In a further embodiment, the formulation is stable for at least about 12 months. In a further embodiment, the formulation is stable for at least about 18 months. In a further embodiment, the formulation is stable for at least about 24 months. In a further embodiment, there is <10% decrease in cannabinoid content. In a further embodiment, there is <5% decrease in cannabinoid content.

In a further embodiment, the composition is stable at about 40° C.±2° C./75% RH±5% RH for a period of time that is at least about 2 months, with <20% decrease in active ingredient, e.g., in cannabinoid content and no change on dispersion in 37° C. water over the period of time. In a further embodiment, the formulation is stable for at least about 9 months. In a further embodiment, the formulation is stable for at least about 12 months. In a further embodiment, the formulation is stable for at least about 24 months. In a further embodiment, there is <10% decrease in cannabinoid content. In a further embodiment, there is <5% decrease in cannabinoid content.

It is preferred that the composition of the present invention is stable for at least 6 months after opening of the packaging in which the composition is provided to a subject. Many oil-containing oral solutions of cannabinoids provide instructions that the contents must be disposed of 12 weeks after opening. This is due to degradation of the cannabinoids in the oil, and/or contamination of the oil by repeated insertion of dispensing syringes.

Without being held to any theory, it is believed that the stability of the composition of the present invention is due to one or more of the following:
the presence of an antioxidant;
the presence of a PEG ester; and/or
the absence of an oil carrier.

There is provided a composition comprising a cannabinoid and a PEG ester wherein the composition further comprises an antioxidant selected from the list comprising: butylated hydroxytoluene (BHT), butylated hydroxyanisole (BHA), ascorbyl palmitate, alpha-tocopherol, sodium metabisulfite, propyl gallate, cysteine, and combinations thereof. Preferable the antioxidant is selected from the list comprising: BHT, BHA or combinations thereof.

Preferably compositions of the present invention do not comprise an oil, or contain only a minimal amount of an oil or lipid. Preferably compositions of the present invention do not contain an oil or lipid chosen from: sesame seed oil, bran oil, coconut oil, palm kernel oil or olive oil, or contain a minimal amount of such an oil or lipid.

### Food Effects

It is known that eating food, particularly fatty or oily food, can increase the amount of orally delivered cannabinoid absorbed by the gut. However, this is a variable and unpredictable effect, depending on the type and amount of food, and the timing of food consumption with respect to drug dosing (Silmore et al (2021) Pharmacotherapy 41:405-420). As stated in Silmore et al (2021), “an oil volume as low as 2 milliliters has been reported to stimulate increased bile salt secretion, elevate levels of phospholipid and cholesterol in the intestine, and trigger gall bladder contraction. Thus, CBD drug product excipients are likely contributing to the increased interindividual variability in fasted state studies-especially with higher doses as greater oil volumes may largely affect the gastrointestinal mechanisms in digestion as the high doses are administered in conjunction with large amounts of sesame oil vehicle”.

The presence of a PEG ester in combination with a cannabinoid reduces the effect that food has on the bioavailability of the cannabinoid. The presence of the PEG ester renders the cannabinoid highly bioavailable as it is maximally dispersed in the aqueous gut liquid. The bioavailability of the cannabinoid in the presence of a PEG ester does not increase significantly in the presence or absence of food, particularly fatty or oily food, as it is already highly bioavailable.

The compositions of the present invention that comprise a cannabinoid and a PEG ester preferably do not have to be taken with food. For example, the compositions of the present invention can be taken in a fasting state. Furthermore, the compositions of the present invention may be taken by subjects on calorie-controlled diets.

Preferably the fed state of the subject being administered the compositions of the present invention changes the Cₘₐₓ by less than a 5-fold change. For example, the fed state of the subject may change the Cₘₐₓ by less than a 4-fold change, 3-fold change, or 2-fold change. Thus, the Cₘₐₓ of a subject when administered the composition in a fed state is less than 5-fold more than the Cₘₐₓ of a subject administered the composition in a fasting state.

Preferably the fed state of the subject being administered the compositions of the present invention changes the AUC₍₀₋₂₄ₕ₎ by less than a 5-fold change. For example, the fed state of the subject may change the AUC₍₀₋₂₄ₕ₎ by less than a 4-fold change, 3-fold change, or 2-fold change. Thus, the AUC₍₀₋₂₄ₕ₎ of a subject when administered the composition in a fed state is less than 5-fold more than the AUC₍₀₋₂₄ₕ₎ of a subject administered the composition in a fasting state.

Many commercial cannabinoid compositions comprise an oil. Whilst the amount of oil may not be significant, if s subject is on a calorie-controlled diet, the addition of these calories may be a problem. For example, daily doses of Epidiolex™ CBD oil may contain between 1.4 mL and 44 mL of oil (Silmore et al (2021)).

Preferably administration of the compositions of the present invention delivers fewer calories to the subject being administered compositions than a comparator composition that comprise an oil or lipid, or comparator compositions that comprise more than a minimal amount of an oil or lipid. Preferably administration of the compositions of the present invention delivers fewer than half the calories per mg of cannabinoid in the composition than a comparator composition (i.e. if a subject takes enough comparator composition to give a dose of 150 mg cannabinoid per day, taking the present composition at 150 mg cannabinoid per day will deliver fewer than half the additional calories).

### Side Effects

Many compositions of the prior art that comprise an oil and/or a high dose of cannabinoid are associated with treatment-emergent adverse event (TEAE). The adverse side effects are often gastrointestinal (GI) associated effects. For example, the GI-associated effects may include: diarrhoea, nausea, constipation, bloating, stomach cramp, decreased appetite, or other GI effects.

The compositions of the present invention preferably have reduced TEAE, particularly reduced GI-associated side effects. Without being held to any theory, it is believed that the side effects may be due at least in part to the presence of oil in prior art cannabinoid compositions (particularly sesame oil which is an allergen and/or can induced food intolerance in many subjects). The present invention seeks to avoid such oil-induced side effects by reducing or removing oil from the compositions and the PEG esters of the present invention were selected as excipients to avoid the use of oil or at least reduce its presence in oral compositions.

It is known that a cannabinoid mixed with an oil may precipitate out of the oil in presence of water; such precipitation may result in the formation of lipophilic oily droplets in the gut, thus lowering the absorption as such lipophilic oily droplets are poorly absorbed by the gut. The PEG esters of the present invention were selected to hold the cannabinoids in a dissolved form due to their miscibility with both water and lipophilic cannabinoids.

If more of the cannabinoid is available for absorption due to the miscibility with water provided by the PEG esters, it might be expected that such compositions would induce “dose dumping”. More of the cannabinoid may be available earlier or at one time, and thus a large load of cannabinoid may enter the blood at once. Dose dumping can be associate with GI-associated side effects as a potentially toxic quantity of the drug is introduced into the systemic circulation. Large doses of cannabinoids are known to produce GI effects and the presence of the PEG esters in combination with a cannabinoid may be expected to simulate the effect of high dose cannabinoids by increasing the rate and amount of active absorbed.

However, the present invention has surprisingly found that compositions comprising PEG esters provide a slower plasma curve to Cₘₐₓ than compositions that do not comprise a PEG ester and/or do comprise significant amounts of oil. That is the compositions comprising PEG esters surprisingly do not induce dose dumping.

As a result of the avoidance of dose dumping provided by the compositions of the present invention, the compositions are able to reduce or avoid the GI-associated side effects of compositions that do not comprise a PEG ester.

An example of TEAE GI-associated effects from commercial oil-containing CBD formulations can be found in the applications for EMA approval of Epidiolex™ for treatment of:
Lennox-Gastaut Syndrome/Dravet Syndrome (LGS/DS population)
[https://www.ema.europa.eu/en/documents/assessment-report/epidyolex-epar-public-assessment-report_en.pdf]
Tuberous Sclerosis Complex (TSC population)
[https://www.ema.europa.eu/en/documents/variation-report/epidyolex-h-c-4675-ii-0005-epar-assessment-report-variation_en.pdf].

In the EMA LGS/DS Assessment Report, a TEAE dose-dependent decrease of appetite (22% in 20 mg/kg/day CBD groups) was observed, which in some patients translated into a weight loss (11% in 20 mg/kg/day CBD groups). In the EMA TSC Assessment Report, the TEAE of diarrhoea was far more common in the subjects administered 25-50 mg/kg/day for treatment of Tuberous Sclerosis Complex (TSC population) as compared to the subjects administered 5-20 mg/kg/day in the Lennox-Gastaut Syndrome/Dravet Syndrome (LGS/DS population). 30.7-56.2% of those on 25-50 mg/kg/day reported a TEAE of diarrhoea, while 12.9-21.2% of those on 5-20 mg/kg/day reported a TEAE of diarrhoea.

The present invention therefore provides a composition comprising a cannabinoid and a PEG ester that induces less TEAE than an oral pharmaceutical composition which comprises the same amount of cannabinoid but does not comprise a PEG ester. More particularly, the invention provides a composition comprising a cannabinoid and a PEG ester that induces less GI-associated TEAE than an oral pharmaceutical composition which comprises the same amount of cannabinoid but does not comprise a PEG ester.

For example, the composition of the present invention may induce a TEAE incidence of less than 20% when administered at 5-20 mg/kg/day, less than 15%, less than 10%, less than 5%, less than 2% or less than 1% when administered at 5-20 mg/kg/day. The composition of the present invention may induce a TEAE incidence of less than 35% when administered at 25-50 mg/kg/day, less than 30%, less than 25%, less than 20%, less than 15%, less than 10% or less than 5% when administered at 25-50 mg/kg/day.

### Method of Treatment

The pharmaceutical compositions of the present invention may be used to treat, ameliorate or prevent a range of diseases and conditions that are responsive to the administration of cannabinoids. For example, the compositions of the present invention may be used to treat a central nervous system (CNS) disease or condition, or an inflammation related disease or condition.

The CNS disease or condition may be, for example, headache, migraine, seizure disorders (including epilepsy), autism, dizziness, attention deficit/hyperactivity disorder (ADHD), Tourette's, insomnia, chronic pain or neuralgia or fibromyalgia.

Preferably the seizure disorder is classified as:
1. Unknown cause (mostly genetic or presumed genetic origin)
   a. Pure epilepsies due to single gene disorders
   b. Pure epilepsies with complex inheritance
2. Symptomatic (associated with gross anatomic or pathologic abnormalities)
   a. Mostly genetic or developmental causation
      i. Childhood epilepsy syndromes
      ii. Progressive myoclonic epilepsies
      iii. Neurocutaneous syndromes
      iv. Other neurologic single gene disorders
      v. Disorders of chromosome function
      vi. Developmental anomalies of cerebral structure
   b. Mostly acquired causes
      i. Hippocampal sclerosis
      ii. Perinatal and infantile causes
      iii. Cerebral trauma, tumor or infection
      iv. Cerebrovascular disorders
      v. Cerebral immunologic disorders
      vi. Degenerative and other neurologic conditions
3. Provoked (a specific systemic or environmental factor is the predominant cause of the seizures)
   a. Provoking factors
   b. Reflex epilepsies
4. Cryptogenic

Preferably, the childhood epilepsy syndrome is chosen from the list comprising:
Childhood Epilepsy with Centrotemporal Spikes
Childhood Absence Epilepsy (CAE)
Early Onset Occipital Epilepsy (Panayiotopoulos Syndrome)
Epilepsy with Myoclonic Atonic Seizures (EMAS)
Dravet Syndrome
Early Myoclonic Encephalopathy (EME)
Juvenile Absence Epilepsy (JAE)
Juvenile Myoclonic Epilepsy (JME)
Landau-Kleffner Syndrome (LKS)
Lennox-Gastaut Syndrome (LGS)
Reflex Epilepsies
Rasmussen's Syndrome
West's Syndrome (Infantile Spasms)
Rett Syndrome

More preferably the seizure disorder is chosen from the list comprising: Lennox-Gastaut syndrome (LGS), Dravet syndrome (DS) and also Tuberous sclerosis complex (TSC).

The CNS disease or condition may be, for example, mental health disorders such as treatment resistant depression; anxiety disorders including Social Anxiety Disorder, Generalised Anxiety Disorders; Post-Traumatic Stress Disorder; symptoms of psychological distress.

The inflammation related disease or condition may be, for example, arthritis such as rheumatoid arthritis and osteoarthritis, multiple sclerosis, irritable bowel syndrome and inflammatory bowel disease (IBD), cancer and cancer-related symptoms.

The present invention therefore provides a method for the treatment, amelioration or prevention of a condition responsive to administration of a cannabinoid, said method comprising the step of:
a) administering a pharmaceutical composition comprising:
i. a cannabinoid;
ii. a polyethylene glycol (PEG) ester.

Preferably the pharmaceutical composition does not compromise an oil or lipid, or only compromises a minimal amount of an oil or lipid.

Preferably the pharmaceutical composition comprises a ratio of from 1:5 w/w cannabinoid:PEG ester to 2:3 w/w cannabinoid:PEG ester.

The present invention further provides a method for the treatment, amelioration or prevention of a condition responsive to administration of a cannabinoid, said method comprising the step of:
a) administering a pharmaceutical composition comprising:
   i. a cannabinoid;
   ii. a polyethylene glycol (PEG) ester
      wherein the bioavailability of the pharmaceutical composition comprising a polyethylene glycol (PEG) ester is from 0.8 to 12 times higher than the bioavailability of an oral pharmaceutical composition which comprises the same amount of cannabinoid but does not comprise a polyethylene glycol (PEG) ester.

The present invention further provides a method for the treatment, amelioration or prevention of a condition responsive to administration of a cannabinoid, said method comprising the step of:
a) administering a pharmaceutical composition comprising:
   i. a cannabinoid;
   ii. a polyethylene glycol (PEG) ester
      wherein the prophylactically or therapeutically effective amount of the pharmaceutical composition comprising a polyethylene glycol (PEG) ester is 0.08 to 1.25 times the prophylactically or therapeutically effective amount of an oral pharmaceutical composition which comprises the same amount of cannabinoid but does not comprise a polyethylene glycol (PEG) ester.

Those skilled in the art will appreciate that the term “dosage form” or “unit dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

The exact regimen for administration of the cannabinoid may depend on the needs of the individual subject being treated, the type of treatment administered, and/or the judgment of the attending medical specialist. As used herein, the terms “subject” and “patient” includes both humans and animals. In some embodiments, the subject or patient is a human adult, human adolescent, human child, or human infant. As those skilled in the art will appreciate, the dosage administered will depend upon the condition being treated, the age, health and weight of the recipient, the type of concurrent treatment, if any, and the frequency of treatment.

The present compositions may also be tailored such that a therapeutically effective amount of the total active ingredient(s) is personalized. In this context, the term “personalized dose” refers to a method wherein the therapeutically effective dose is tailored to an individual patient, based on the individual's predicted response and alleviation of symptoms of a disease and other consideration. Diagnostic testing can be employed for selecting appropriate and optimal therapies based on the context of a patient's molecular or biochemical analyses and other personalization measures (potentially including genetics).

In some embodiments, a composition or a pharmaceutical composition comprising a cannabinoid may be administered in a therapeutically effective amount. A therapeutically effective amount may be administered according to a dosing regimen comprising one or more unit doses. Generally, a therapeutically effective amount is sufficient to achieve a benefit to the subject (e.g., prophylaxis, treating, modulating, curing, preventing and/or ameliorating a disease or condition).

Generally, the term “therapeutically effective amount” refers to an amount of the active ingredient that induces a change in a condition treated by a dosage form of the invention, as measured by relevant definition criteria. This may be monitored in an animal model or in a clinical setting. In this sense, the therapeutic effect is also a pharmacodynamic effect. A therapeutically effective amount (and/or unit dose) of a composition for any particular subject may depend upon a variety of factors including the disease or condition being treated; disease or condition severity; the activity of the specific composition employed; the specific composition employed; the age; body weight; fitness; comorbid conditions (e.g., other than the diseases or condition(s) being treated) general health; sex; and diet of the patient; personal history; genetic characteristic; lifestyle parameter; severity of cardiac defect and/or level of risk of cardiac defect; the time of administration; route of administration; concomitant treatments or medications; and/or rate of excretion or metabolism of the specific composition employed; the duration of the treatment; combinations thereof; as well as other factors well known in the medical arts. In view of the present disclosure, one of ordinary skill in the art will be readily able to determine appropriate dosages depending on these and other related factors. In addition, both objective and subjective assays may optionally be employed to identify optimal dosage ranges. In some particular embodiments, appropriate doses or amounts to be administered may be extrapolated in view of the instant disclosure from dose-response curves derived from in vitro or animal model test systems.

A therapeutically effective amount (also pharmacologically or pharmaceutically or physiologically effective amount) also means an amount of active ingredient (a cannabinoid or a combination) that is needed to provide a desired level of active agent in the bloodstream or at a target organ or to provide an anticipated physiological response. The precise amount will depend upon numerous factors, e.g. type of an agent, activity of a composition, intended patient use (e.g. number of doses per day), patient considerations, and others, which can readily be determined by one skilled in the art. An effective amount of an agent can be determined using standard clinical procedures for determining appropriate amounts and timing of administration. It is understood that the effective amount can be the result of empirical and/or individualized (case-by-case) determination on the part of the treating health care professional and/or individual.

The present invention contemplates dosing regiments comprising single as well as multiple administrations of a composition as described. The composition can be administered at regular intervals, depending on the nature, severity and extent of the subject's condition. In some embodiments, the composition may be administered periodically at regular intervals (e.g., once every year, once every six months, once every five months, once every three months, bimonthly (once every two months), monthly (once every month), biweekly (once every two weeks), weekly, daily, multiple times each day, or continuously).

A therapeutically effective amount of a cannabinoid may be administered according to a dosing regimen that may comprise multiple unit doses. For any particular composition, a therapeutically effective amount (and/or an appropriate unit dose within an effective dosing regimen) may vary, for example, depending on route of administration, or combination with other pharmaceutical agents.

In some embodiments, the composition as described may be administered as a single dose. In some embodiments, a composition or a pharmaceutical composition as described may be administered at regular intervals. Administration at an “interval,” as used herein, indicates that the therapeutically effective amount is administered periodically (as distinguished from a one-time dose). The interval can be determined by standard clinical techniques. In some embodiments, the composition as described may be administered bimonthly, monthly, twice monthly, triweekly, biweekly, weekly, twice weekly, thrice weekly, daily, twice daily, every six hours, every four hours, every two hours, or hourly. The administration interval for a given individual need not be a fixed interval, but may be varied over time, depending on the needs of the individual.

In some embodiments, the composition as described is administered at regular intervals indefinitely. In some embodiments, the composition is administered at regular intervals for a defined period.

It is to be further understood that for any particular subject, specific dosage regimens should be adjusted over time according to the individual need and the professional judgment of the person administering or supervising the administration of the cannabidiol and that dosage ranges set forth herein are exemplary only and are not intended to limit the scope or practice of the claimed invention.

As used herein, “improve”, “increase”, “inhibit” or “reduce”, or grammatically comparable comparative terms, indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

In some embodiments, a change in the condition being treated is identified if there is at least 5% improvement, or 10% improvement, or at least 25%, or at least 50%, or at least 75%, or at least 100% improvement. The change can be based on improvements in the severity of the treated condition in an individual, or on a difference in the frequency of improved conditions in populations of subjects with and without treatment with dosage forms of the invention, or with dosage forms of the invention in combination with other drugs.

“Prevent or prevention” as used herein refers to reducing the risk of developing the disease or condition and/or to delaying onset of one or more characteristics or symptoms of the disease or condition. Prevention may be considered complete when onset of the disease or condition has been delayed for a predefined period of time.

According to the present invention, “symptoms are reduced” when one or more symptoms of the disease or condition is reduced in magnitude (e.g., intensity, severity, etc.) and/or frequency. For purposes of clarity, a delay in the onset of a particular symptom is considered one form of reducing the frequency of that symptom.

As used herein, the term “treat”, “treatment”, or “treating” refers to any method used to partially or completely alleviate, ameliorate, relieve, inhibit, prevent, delay onset of, reduce severity of, and/or reduce incidence of one or more symptoms or features of the disease or condition. In some embodiments, treatment refers to administration of a therapy that partially or completely alleviates, ameliorates, relives, inhibits, delays onset of, reduces severity of, and/or reduces incidence of one or more symptoms, features, and/or causes of the disease or condition. Treatment may be administered to a subject who does not exhibit signs of the disease or condition. In some embodiments, treatment may be administered to a subject who exhibits only early signs the disease or condition, for example for the purpose of decreasing the risk of developing pathology associated with the disease or condition.

As used herein, the term “subject” or “patient” refers an organism, typically a mammal (e.g., a human). In some embodiments, a subject or patient refers to any organism (e.g., mammals such as mice, rats, rabbits, non-human primates, and humans; insects; worms; etc.) to which a provided compound or composition is administered in accordance with the present disclosure e.g., for experimental, diagnostic, prophylactic, and/or therapeutic purposes. In some embodiments, a subject is suffering from the disease or condition. In some embodiments, a subject has symptoms associated with the disease or condition. In some embodiments, a subject is susceptible to the disease or condition or symptoms associated with the disease or condition. In some embodiments, a subject displays one or more symptoms or characteristics of the disease or condition. In some embodiments, a subject does not display any symptoms or characteristics of the disease or condition. In some embodiments, a subject is someone with one or more features characteristic of susceptibility to or risk of the disease or condition. In some embodiments, a subject is a patient. In some embodiments, a subject is an individual to whom diagnosis and/or therapy is and/or has been administered. “Individual”, “patient” and “subject” are used interchangeably.

### Delivery

In one embodiment of the invention, the cannabinoid is administered to the subject using a dosing regimen selected from the group consisting of: three times daily; two times daily; daily; every second day, every third day, once weekly; once fortnightly and once monthly.

In accordance with certain embodiments, the composition is administered regularly until treatment is obtained. In one preferred embodiment, the composition is administered to the subject in need of such treatment using a dosing regimen selected from the group consisting of: every hour, every 2 hours, every 3 hours, once daily, twice daily, three times daily, four times daily, five times daily, once weekly, twice weekly, once fortnightly and once monthly. However, other application schedules may be utilized in accordance with the present invention. Preferably, the composition of the treatment regimen is administered to the subject from 1 to 5 times per day, more preferably once or twice per day.

The mode of administration is preferably suitable for the form in which the composition has been prepared. The mode of administration for the most effective response may be determined empirically and the means of administration described are given as examples, and do not limit the method of delivery of the composition of the present invention in any way. All the present compositions are commonly used in the pharmaceutical industry and are commonly known to suitably qualified practitioners.

Preferably the pharmaceutical composition of the present invention is delivered via the oral, rectal or vaginal route. Most preferably, the pharmaceutical composition of the present invention is delivered via the oral route. Therefore, the pharmaceutical composition of the present invention is most preferably an oral pharmaceutical composition. However, pharmaceutical compositions delivered to the vagina or rectum as capsules are contemplated.

The compositions of the invention may optionally include pharmaceutically acceptable nontoxic excipients and carriers. As used herein, a “pharmaceutical carrier” is a pharmaceutically acceptable solvent, suspending agent, excipient or vehicle for delivering the cannabinoid, polyethylene glycol (PEG) ester and any other ingredient to the subject. The carrier may be liquid or solid and is selected with the planned manner of administration in mind.

The composition of the invention may be selected from the group consisting of: an immediate release composition, a delayed release composition, a controlled release composition and a rapid release composition.

### Excipients

The compositions of the present invention may contain excipients to aid in the delivery of the cannabinoid.

The compositions described herein may be formulated by including the active agent in an oil-in-water emulsion, or a water-in-oil emulsion. In such a composition, the immediate release dosage form is in the continuous phase, and the delayed release dosage form is in a discontinuous phase. The composition may also be produced in a manner for delivery of three dosage forms as hereinabove described. For example, there may be provided an oil-in-water-in-oil emulsion, with oil being a continuous phase that contains the immediate release component, water dispersed in the oil containing a first delayed release dosage form, and oil dispersed in the water containing a third delayed release dosage form.

The compositions described herein may be in the form of a liquid composition. The liquid composition may comprise a solution that includes an active agent dissolved in a solvent. Generally, any solvent that has the desired effect may be used in which the active agent dissolves and which can be administered to a subject. Generally, any concentration of active agent that has the desired effect can be used. The composition in some variations is a solution which is unsaturated, a saturated or a supersaturated solution. The solvent may be a pure solvent or may be a mixture of liquid solvent components. In some variations the solution formed is an in-situ gelling composition. Solvents and types of solutions that may be used are well known to those versed in such drug delivery technologies.

The composition may or may not contain water. Preferably, the composition does not contain water, i.e. it is non-aqueous. In another preferred embodiment, the composition does not comprise a preservative.

The administration of the cannabinoids in accordance with the methods and compositions of the invention may be by any suitable means that results in an amount sufficient to treat the disease or condition.

The cannabinoid may be contained in any appropriate amount and in any suitable carrier substance and is generally present in an amount of 1-95% by weight of the total weight of the composition.

The pharmaceutical composition may be formulated according to the conventional pharmaceutical or veterinary practice (see, for example, Remington: The Science and Practice of Pharmacy, 20th edition, 2000, ed; A. R. Gennaro, Lippincott Williams & Wilkins, Philadelphia, and Encyclopedia of Pharmaceutical Technology, eds; J. Swarbrick and J. C. Boylan, 1988-1999, Marcel Dekker, New York; Remington's Pharmaceutical Sciences, 18^{th} Edition, Mack Publishing Company, Easton, Pennsylvania, USA).

Generally, examples of suitable carriers, excipients and diluents include, without limitation: water, saline, ethanol, dextrose, glycerol, lactose, dextrose, sucrose, sorbitol, mannitol, starches, gum acacia, calcium phosphates, alginate, tragacanth, gelatine, calcium silicate, microcrystalline cellulose, polyvinylpyrrolidone, cellulose, water syrup, methyl cellulose, methyl and propylhydroxybenzoates, polysorbates, talc magnesium stearate, mineral oil or combinations thereof. The compositions can additionally include lubricating agents, pH buffering agents, wetting agents, emulsifying and suspending agents, preserving agents, sweetening agents or flavouring agents.

The composition may be in the form of a controlled-release composition and may include a degradable or non-degradable polymer, hydrogel, organogel, or other physical construct that modifies the release of the cannabinoid. It is understood that such compositions may include additional inactive ingredients that are added to provide desirable colour, stability, buffering capacity, dispersion, or other known desirable features. Such compositions may further include liposomes, such as emulsions, foams, micelles, insoluble monolayers, liquid crystals, phospholipid dispersions, lamellar layers and the like. Liposomes for use in the invention may be formed from standard vesicle-forming lipids, generally including neutral and negatively charged phospholipids and a sterol, such as cholesterol.

Contemplated for use herein are oral solid dosage forms, which are described generally in Martin, Remington's Pharmaceutical Sciences, 18th Ed. (1990 Mack Publishing Co. Easton PA 18042) at Chapter 89, which is herein incorporated by reference. Solid dosage forms include tablets, capsules, pills, troches or lozenges, cachets or pellets. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatised with various polymers (E.g., U.S. Pat. No. 5,013,556). A description of possible solid dosage forms for the therapeutic is given by Marshall, in Modern Pharmaceutics, Chapter 10, Banker and Rhodes ed., (1979), herein incorporated by reference. In general, the composition will include therapeutic agents (e.g. cannabinoid), and inert ingredients which allow for protection against the stomach environment, and release of the cannabinoid in the intestine.

For the cannabinoid in the pharmaceutical composition of the invention, the location of release may be the stomach, the small intestine (the duodenum, the jejunum, or the ileum), or the large intestine. One skilled in the art has available compositions that will not dissolve in the stomach, yet will release the material in the duodenum or elsewhere in the intestine. In one aspect, the release will avoid the deleterious effects of the stomach environment, either by protection of the composition or by release of the cannabinoid beyond the stomach environment, such as in the intestine.

It is believed that the oral bioavailability of cannabinoids is only 4% to 12% and absorption is highly variable. Although most cannabinoids are generally easily absorbed due to their high partition coefficient (P), they are subject to degradation in the stomach and significant first-pass metabolism. Therefore, preferably, the cannabinoid of the present invention is released in the lower gastrointestinal tract.

The oral dosage method may be provided using an oral sustained release pharmaceutical composition comprising a therapeutically effective pharmaceutical composition according to the invention, and a release retardant.

In one aspect of the present invention the release retardant is a water-soluble, water swellable and/or water insoluble polymer. In particular, water-soluble polymers may be selected from the group comprising ethylcellulose, hydroxypropyl cellulose, hydroxypropyl methyl cellulose. The release retardant may be an enteric coating or a semipermeable membrane. In another aspect of the invention the release retardant is a non-polymeric release retardant. More particularly, the non-polymeric release retardant may be hydrogenated castor oil. The compositions of the invention may be milled or granulated and compressed into tablets or encapsulated into capsules according to conventional procedures known in the art.

To ensure full gastric resistance, a coating impermeable to at least pH 5.0 is used. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This includes without limitation sugar coatings, or coatings that make the tablet easier to swallow. Exemplary capsules include hard shell capsules (such as gelatine) for delivery of dry therapeutics such as powders, or soft gelatine shells for liquid therapeutic forms. The shell material of cachets in certain aspects is thick starch or other edible paper. For pills, lozenges, moulded tablets or tablet triturates, moist massing techniques are also contemplated, without limitation.

As used herein, the term “sustained release” means the gradual but continuous or sustained release over a relatively extended period of the therapeutic compound content after oral ingestion. The release may continue after the pharmaceutical composition has passed from the stomach and through until and after the pharmaceutical composition reaches the intestine. The phrase “sustained release” also means delayed release wherein release of the therapeutic compound is not immediately initiated upon the pharmaceutical composition reaching the stomach but rather is delayed for a period of time, for example, until when the pharmaceutical composition reaches the intestine. Upon reaching the intestine, the increase in pH may then trigger release of the therapeutic agent from the pharmaceutical composition.

Though term “release retardant” is used herein, means a substance that reduces the rate of release of a therapeutic agent from a pharmaceutical composition when orally ingested. The release retardant may be a polymer or a non-polymer. The release retardant may be used according to any one of several sustained release systems including, for example, a diffusion system, a dissolution system and/or an osmotic system.

However, as well as oral compositions, rectally and vaginally delivered compositions are contemplated. These compositions may also comprise coatings, release retardants, water swellable and/or water insoluble polymers, and other routine pharmaceutical excipients.

In certain aspects, the therapeutic agent (e.g. cannabinoid) is included in the composition as fine multi-particulates in the form of granules or pellets of particle size about 1 mm. The composition of the material for capsule administration is, in certain aspects, a powder, lightly compressed plug, or even as a tablet. In one aspect, the composition comprising the therapeutic agent could be prepared by compression.

Colourants and flavouring agents may optionally be included. For example, compositions may be formulated (such as, and without limitation, by liposome or microsphere encapsulation) and then further contained within an edible product, such as a refrigerated beverage containing colorants and flavouring agents.

The volume of the composition may be diluted or increased with an inert material. These diluents could include carbohydrates, especially mannitol, alpha-lactose, anhydrous lactose, cellulose, sucrose, modified dextrans and starch. Certain inorganic salts are also optionally used as fillers including calcium triphosphate, magnesium carbonate and sodium chloride. Some commercially available diluents are Fast-Flo™, Emdex™, STA-Rx™ 1500, Emcompress™ and Avicell™.

In other embodiments, disintegrants are included in the solid dosage form compositions of the present invention. Materials used as disintegrants include but are not limited to starch including the commercial disintegrant based on starch, Explotab™. Sodium starch glycolate, Amberlite™, sodium carboxymethylcellulose, ultramylopectin, sodium alginate, gelatine, orange peel, acid carboxymethyl cellulose, natural sponge and bentonite are also contemplated. Another form of the disintegrants is the insoluble cationic exchange resins. Powdered gums are also optionally used as disintegrants and as binders and these include, without limitation, powdered gums such as agar, karaya or tragacanth. Alginic acid and its sodium salt are also useful as disintegrants.

Binders are contemplated to hold the cannabinoid together to form a hard tablet and include, without limitation, materials from natural products such as acacia, tragacanth, starch and gelatine. Other binders include, without limitation, methylcellulose (MC), ethyl cellulose (EC) and carboxymethyl cellulose (CMC). Polyvinyl pyrrolidone (PVP) and hydroxypropylmethyl cellulose (HPMC) are contemplated for use in alcoholic solutions to granulate the therapeutic.

An antifrictional agent may be optionally included in the compositions of the invention to prevent sticking during the composition process. Lubricants may be optionally used as a layer between the therapeutic and the die wall, and these can include but are not limited to: stearic acid including its magnesium and calcium salts, polytetrafluoroethylene (PTFE), liquid paraffin, vegetable oils and waxes. Exemplary soluble lubricants may also be used such as include sodium lauryl sulfate, magnesium lauryl sulfate, polyethylene glycol of various molecular weights, and Carbowax 4000 and 6000.

Glidants that might improve the flow properties of the compound during composition and to aid rearrangement during compression might be optionally added. The glidants may include without limitation starch, talc, pyrogenic silica and hydrated silicoaluminate.

To aid dissolution of the therapeutic agent (e.g. cannabinoid) into the aqueous environment, a surfactant might be added in certain embodiments as a wetting agent. Surfactants may include, for example and without limitation, anionic detergents such as sodium lauryl sulfate, dioctyl sodium sulfosuccinate and dioctyl sodium sulfonate. Cationic detergents might be optionally used and could include, without limitation, benzalkonium chloride or benzethomium chloride. The list of potential nonionic detergents that could be included in the composition as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. When used, these surfactants could be present in the composition of the therapeutic agent either alone or as a mixture in different ratios.

Additives which that potentially enhance uptake of the therapeutic agent include, without limitation, the fatty acids oleic acid, linoleic acid and linolenic acid.

Controlled release composition may be desirable. In certain aspects, the therapeutic agents could be incorporated into an inert matrix that permits release by either diffusion or leaching mechanisms i.e., gums. In some aspects, slowly degenerating matrices may also be incorporated into the composition. Another form of a controlled release of this therapeutic is by a method based on the Oros therapeutic system (Alza Corp.), i.e. the drug is enclosed in a semipermeable membrane which allows water to enter and push drug out through a single small opening due to osmotic effects. Some enteric coatings also have a delayed release effect.

### Use

The present invention provides for the use of:
a) a cannabinoid; and
b) a polyethylene glycol (PEG) ester
   for the manufacture of a pharmaceutical composition for the treatment, amelioration or prevention of a condition responsive to administration of a cannabinoid.

Preferably the pharmaceutical composition does not compromise an oil or lipid, or only compromises a minimal amount of an oil or lipid.

Preferably the pharmaceutical composition comprises a ratio of from 1:5 w/w cannabinoid:PEG ester to 2:3 w/w cannabinoid:PEG ester.

The present invention provides for the use of:
a) a cannabinoid; and
b) a polyethylene glycol (PEG) ester
   for the manufacture of a pharmaceutical composition for the treatment, amelioration or prevention of a condition responsive to administration of a cannabinoid, wherein the bioavailability of the pharmaceutical composition comprising a polyethylene glycol (PEG) ester is from 0.8 to 12 times higher than the bioavailability of an oral pharmaceutical composition which comprises the same amount of cannabinoid but does not comprise a polyethylene glycol (PEG) ester.

The present invention further provides for the use of:
a) a cannabinoid; and
b) a polyethylene glycol (PEG) ester
   for the manufacture of a pharmaceutical composition for the treatment, amelioration or prevention of a condition responsive to administration of a cannabinoid, wherein the prophylactically or therapeutically effective amount of the pharmaceutical composition comprising a polyethylene glycol (PEG) ester is from 0.08 to 1.25 times the prophylactically or therapeutically effective amount of an oral pharmaceutical composition which comprises the same amount of cannabinoid but does not comprise a polyethylene glycol (PEG).

The present invention provides for the use of a composition comprising:
a) a cannabinoid; and
b) a polyethylene glycol (PEG) ester
   for the treatment, amelioration or prevention of a condition responsive to administration of a cannabinoid.

The present invention further provides for the use of a pharmaceutical composition comprising:
a) a cannabinoid; and
b) a polyethylene glycol (PEG) ester
   for the treatment, amelioration or prevention of a condition responsive to administration of a cannabinoid, wherein the bioavailability of the pharmaceutical composition comprising a polyethylene glycol (PEG) ester is from 0.8 to 12 times higher than the bioavailability of an oral pharmaceutical composition which comprises the same amount of cannabinoid but does not comprise a polyethylene glycol (PEG) ester.

The present invention further provides for the use of a pharmaceutical composition comprising:
a) a cannabinoid; and
b) a polyethylene glycol (PEG) ester
   for the treatment, amelioration or prevention of a condition responsive to administration of a cannabinoid, wherein the prophylactically or therapeutically effective amount of the pharmaceutical composition comprising a polyethylene glycol (PEG) ester is from 0.08 to 1.25 times the prophylactically or therapeutically effective amount of an oral pharmaceutical composition which comprises the same amount of cannabinoid but does not comprise a polyethylene glycol (PEG).

### Kits

The present invention provides a kit for the treatment, amelioration or prevention of a condition responsive to administration of a cannabinoid, said kit comprising:
a) a pharmaceutical composition comprising:
   i. a cannabinoid; and
   ii. a polyethylene glycol (PEG) ester
b) instructions for use.

Preferably the pharmaceutical composition does not compromise an oil or lipid, or only compromises a minimal amount of an oil or lipid.

Preferably the pharmaceutical composition comprises a ratio of from 1:5 w/w cannabinoid:PEG ester to 2:3 w/w cannabinoid:PEG ester.

The present invention further provides a kit for the treatment, amelioration or prevention of a condition responsive to administration of a cannabinoid, said kit comprising:
a) a pharmaceutical composition comprising:
   i. a cannabinoid; and
   ii. a polyethylene glycol (PEG) ester
b) instructions for use
   wherein the bioavailability of the pharmaceutical composition comprising a polyethylene glycol (PEG) ester is from 0.8 to 12 times higher than the bioavailability of an oral pharmaceutical composition which comprises the same amount of cannabinoid but does not comprise a polyethylene glycol (PEG) ester.

The present invention further provides a kit for the treatment, amelioration or prevention of a condition responsive to administration of a cannabinoid, said kit comprising:
a) a pharmaceutical composition comprising:
   i. a cannabinoid; and
   ii. a polyethylene glycol (PEG) ester
b) instructions for use
   wherein the prophylactically or therapeutically effective amount of the pharmaceutical composition comprising a polyethylene glycol (PEG) ester is from 0.08 to 1.25 times the prophylactically or therapeutically effective amount of an oral pharmaceutical composition which comprises the same amount of cannabinoid but does not comprise a polyethylene glycol (PEG) ester.

Kits of the invention include one or more containers comprising cannabidiol as described herein, and instructions for use in accordance with any one of the methods described herein. The kit may further comprise a description for selecting a subject suitable for treatment based on identifying whether that individual has symptoms of the disease or condition that is responsive to the administration of cannabinoids. The kit may further comprise a description of administering cannabidiol as described herein to an individual at risk of developing symptoms of the disease or condition.

The composition may be delivered once per day, or more than once per day (for example, twice per day, three times per day or four times per day) for a total daily dose of from 25 mg to 200 mg of cannabidiol.

The instructions generally include information as to dosage, dosing schedule, and route of administration for the intended treatment. The containers may be unit doses, bulk packages (e.g. multi-dose packages) or sub-unit doses. Instructions supplied in the kits of the invention are typically written instructions on a label or package insert. The label or package insert indicates that the composition is used for treating and/or preventing symptoms of the disease or condition. Instructions may be provided for practising any of the methods described herein.

### General

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (eg. Size, displacement and field strength etc). A range of values will be understood to include all values within the range, including the values defining the range, and values adjacent to the range which lead to the same or substantially the same outcome as the values immediately adjacent to that value which defines the boundary to the range. Accordingly, unless indicated to the contrary, the numerical parameters set forth in the specification and claims are approximations that may vary depending upon the desired properties sought to be obtained by the present invention. Hence “about 80%” means “about 80%” and also “80%”. At the very least, each numerical parameter should be construed in light of the number of significant digits and ordinary rounding approaches.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs. The term “active agent” may mean one active agent, or may encompass two or more active agents.

The following examples serve to more fully describe the manner of using the above-described invention, as well as to set forth the best modes contemplated for carrying out various aspects of the invention. It is understood that these methods in no way serve to limit the true scope of this invention, but rather are presented for illustrative purposes.

### EXAMPLES

Further features of the present invention are more fully described in the following non-limiting Examples. This description is included solely for the purposes of exemplifying the present invention. It should not be understood as a restriction on the broad description of the invention as set out above.

### Example 1

### Capsule Formation

TABLE 2
Exemplary capsule dosage forms of the present invention

| Gelucire | | Vitamin E TPGS | | PEG3350 | |
|---|---|---|---|---|---|
| Composition | mg/capsule | Composition | mg/capsule | Composition | mg/capsule |
| Gelucire 5013 | 250 | Vitamin E TPGS | 250 | PEG3350 | 250 |
| Butylated | 0.7 | Butylated | 0.7 | Butylated | 0.7 |
| hydroxytoluene | | hydroxytoluene | | hydroxytoluene | |
| (BHT) | | (BHT) | | (BHT) | |
| Butylated | 0.175 | Butylated | 0.175 | Butylated | 0.175 |
| hydroxyanisole | | hydroxyanisole | | hydroxyanisole | |
| (BHA) | | (BHA) | | (BHA) | |
| Cannabidiol (CBD) | 50 | Cannabidiol (CBD) | 50 | Cannabidiol (CBD) | 50 |

### Example 2

### Pharmacokinetic Study

This study was conducted to evaluate the pharmacokinetics of cannabidiol (CBD) formulations following an oral (capsule or gavage) administration in Beagle dogs.

Eight male beagle dogs were assigned to four different treatments as described in 0. Each dog was administered an oral dose of 50 mg of each test article. A single dose was administered per treatment. Each treatment was separated by a minimum of 7 days.

TABLE 3
Group Assignments

| | | | | | Amount | Total | | |
|---|---|---|---|---|---|---|---|---|
| | | | | | of Test | CBD | | |
| | # of | | | | Article | Dose | Dose | Dose |
| | Animals/ | Route of | Test | Capsules/ | per Capsule | Admin. | Conc. | Volume |
| Treatment | Sex | Admin | Article | Animal | (mg) | (mg) | (mg/mL) | (mL) |
| 1 | 8/M* | PO (gavage) | Epidiolex | N/A | N/A | 50 | 100 | 0.5 |
| 2 | 8/M | PO (capsule) | PEG 3350 | 1 | 50 | 50 | N/A | N/A |
| 3 | 8/M | PO (capsule) | Gelucire | 1 | 50 | 50 | N/A | N/A |
| 4 | 8/M | PO (capsule) | Vitamin E | 1 | 50 | 50 | N/A | N/A |
| | | | TPGS | | | | | |

*The same 8 animals were used for each treatment. Treatments were separated by a minimum 7-day washout interval.
N/A - not applicable.

Twelve serial blood samples (˜3 ml per sample timepoint) per treatment were obtained from each dog via direct venipuncture of a jugular vein using blood collection tubes containing K₂-EDTA as the anticoagulant at the following times (post-dose): 0 min (pre-dose), 10 min, 30 min, 1 hour, 1.5 hours, 2 hours, 2.5 hours, 3 hours, 4 hours, 8 hours, 12 hours, and 24 hours. Collected blood samples were gently inverted several times and stored on chill packs (or ice-water bath) until centrifugation. Blood samples were centrifuged at ˜3000 rpm for 10 min at +4° C. within 30 minutes of collection. Plasma samples were stored frozen at approximately −70° C. or lower.

### Results

Animal body weights and dosing details are summarized in Tables 4-7. All animals appeared healthy at study initiation and throughout the duration of the study. Animals received their intended doses, and test article administration was largely uneventful. A single animal (Dog #1) initially rejected the fourth treatment, resulting in early emesis containing the regurgitated capsule. This occurred approximately four times before the capsule was successfully administered, though capsule remnants were also observed among clear frothy emesis immediately following oral dosing. All blood samples were collected at their targeted times.

TABLE 4
Individual and Mean Animal Body Weights and Dosing Details - Epidolex

| Dog | | Body Wt | Dose Vol | Syringe Weights (g) | | | Dose Level | |
|---|---|---|---|---|---|---|---|---|
| # | Route | (kg) | (ml) | Loaded | Empty | Net | (mg) | (mg/kg) |
| 1 | PO | 8.3 | 0.5 | 3.7859 | 3.2633 | 0.5226 | 52.26 | 6.3 |
| 2 | PO | 11.5 | 0.5 | 3.7350 | 3.2589 | 0.4761 | 47.61 | 4.1 |
| 3 | PO | 9.9 | 0.5 | 3.8420 | 3.3382 | 0.5038 | 50.38 | 5.1 |
| 4 | PO | 9.6 | 0.5 | 3.8185 | 3.3142 | 0.5043 | 50.43 | 5.3 |
| 5 | PO | 9.6 | 0.5 | 3.8275 | 3.3300 | 0.4975 | 49.75 | 5.2 |
| 6 | PO | 9. | 0.5 | 3.7743 | 3.2758 | 0.4985 | 49.85 | 5.5 |
| 7 | PO | 8.4 | 0.5 | 3.7925 | 3.2848 | 0.5077 | 50.77 | 6.0 |
| 8 | PO | 9.7 | 0.5 | 3.7694 | 3.2698 | 0.4996 | 49.96 | 5.2 |
| | Mean | 9.51 | 0.5 | 3.7931 | 3.2919 | 0.5013 | 50.13 | 5.3 |
| | SD | 1.002 | 0.00 | 0.03497 | 0.03116 | 0.01293 | 1.29289 | 0.65 |
| | % RSD | 10.5 | 0.0 | 0.9 | 0.9 | 2.6 | 2.6 | 12.3 |

TABLE 5
Individual and Mean Animal Body Weights
and Dosing Details - PEG3350

| Dog | | Body Wt | Capsule | Dose Level | |
|---|---|---|---|---|---|
| # | Route | (kg) | # | (mg) | (mg/kg) |
| 1 | PO | 8.3 | 1 | 50.00 | 6.0 |
| 2 | PO | 11.4 | 1 | 50.00 | 4.4 |
| 3 | PO | 9.5 | 1 | 50.00 | 5.3 |
| 4 | PO | 9.5 | 1 | 50.00 | 5.3 |
| 5 | PO | 9.5 | 1 | 50.00 | 5.3 |
| 6 | PO | 8.7 | 1 | 50.00 | 5.7 |
| 7 | PO | 8.3 | 1 | 50.00 | 6.0 |
| 8 | PO | 9.3 | 1 | 50.00 | 5.4 |
| | Mean | 9.31 | 1.0 | 50.00 | 5.4 |
| | SD | 0.992 | 0.00 | 0.00000 | 0.53 |
| | % RSD | 10.7 | 0.0 | 0.0 | 9.8 |

TABLE 6
Individual and Mean Animal Body Weights
and Dosing Details - Gelucire

| Dog | | Body Wt | Capsule | Dose Level | |
|---|---|---|---|---|---|
| # | Route | (kg) | # | (mg) | (mg/kg) |
| 1 | PO | 8.4 | 1 | 50.00 | 6.0 |
| 2 | PO | 11.5 | 1 | 50.00 | 4.3 |
| 3 | PO | 9.8 | 1 | 50.00 | 5.1 |
| 4 | PO | 10.0 | 1 | 50.00 | 5.0 |
| 5 | PO | 9.6 | 1 | 50.00 | 5.2 |
| 6 | PO | 8.7 | 1 | 50.00 | 5.7 |
| 7 | PO | 8.4 | 1 | 50.00 | 6.0 |
| 8 | PO | 9.5 | 1 | 50.00 | 5.3 |
| | Mean | 9.49 | 1.0 | 50.00 | 5.3 |
| | SD | 1.029 | 0.00 | 0.00000 | 0.55 |
| | % RSD | 10.8 | 0.0 | 0.0 | 10.3 |

TABLE 7
Individual and Mean Animal Body Weights
and Dosing Details - Vit E TPGS

| Dog | | Body Wt | Capsule | Dose Level | |
|---|---|---|---|---|---|
| # | Route | (kg) | # | (mg) | (mg/kg) |
| 1 | PO | 8.5 | 1 | 50.00 | 5.9 |
| 2 | PO | 11.5 | 1 | 50.00 | 4.3 |
| 3 | PO | 9.9 | 1 | 50.00 | 5.1 |
| 4 | PO | 9.8 | 1 | 50.00 | 5.1 |
| 5 | PO | 9.6 | 1 | 50.00 | 5.2 |
| 6 | PO | 8.9 | 1 | 50.00 | 5.6 |
| 7 | PO | 8.5 | 1 | 50.00 | 5.9 |
| 8 | PO | 9.5 | 1 | 50.00 | 5.3 |
| | Mean | 9.53 | 1.0 | 50.00 | 5.3 |
| | SD | 0.972 | 0.00 | 0.00000 | 0.51 |
| | % RSD | 10.2 | 0.0 | 0.0 | 9.6 |

Sample preparation involved the liquid-liquid extraction of Cannabidiol from 150 μL of dog plasma; Cannabidiol-D3 was used as the internal standard. The compound was identified and quantified using reversed-phase HPLC with MS/MS detection over a theoretical concentration range of 2.00 ng/mL to 2000.00 ng/mL. Concentrations of analyte were determined using the peak area ratio response of the analyte/internal standard with calibration curve linearity established using a weighted (1/×2) linear (y=mx+b) least squares regression analysis.

TABLE 8
Summary of cannabidiol pharmacokinetic parameters following oral
administration of cannabidiol in different formulations in dogs

| | | | Dose | Cₘₐₓ | Tₘₐₓ | AUCₗₐₛₜ | AUC0-ₒₒ | λ_{Z} | T_{1/2} |
|---|---|---|---|---|---|---|---|---|---|
| Analyte | Visit | Test Article | (mg) | (ng/ml) | (hr) | (hr-ng/ml) | (hr-ng/ml) | (1/hr) | (hr) |
| Cannabidiol | Treatment 1 | Epidiolex | 50 | 102.93 | 1.00 | 276.40 | 268.26 | 0.2873 | 3.58 |
| | | | | (93.3) | (0.17-2.50) | (98.8) | (77.4) | (80.6) | (60.9) |
| | | | N | 88 | 8 | 8 | 6 | 6 | 6 |
| Cannabidiol | Treatment 2 | PEG 3350 | 50 | 95.55 (68.3) | 1.75 | 559.89 | 652.42 | 0.1098 | 6.41 |
| | | | | | (1.00-24.00) | (82.3) | (69.4) | (14.2) | (15.0) |
| | | | N | 8 | 8 | 8 | 4 | 4 | 4 |
| Cannabidiol | Treatment 3 | Gelucire | 50 | 124.02 | 1.50 | 540.10 | 634.10 | 0.1351 | 5.29 |
| | | | | (73.8) | (0.50-4.00) | (72.4) | (59.8) | (19.7) | (17.7) |
| | | | N | 8 | 8 | 8 | 7 | 7 | 7 |
| Cannabidiol | Treatment 4 | Vitamin E | 50 | 256.91 | 1.50 | 1139.02 | 1594.06 | 0.1491 | 5.02 |
| | | TPGS | | (73.4) | (1.00-4.00) | (84.0) | (61.3) | (35.8) | (26.3) |
| | | | N | 8 | 8 | 8 | 5 | 5 | 5 |

Data presented as Mean (CV %) except for Tₘₐₓ presented as Median (Minimum-Maximum)

Following a single oral administration of CBD in four treatment groups, Epidiolex (Treatment 1), PEG 3350 (Treatment 2), Gelucire (Treatment 3) and Vitamin E TPGS (Treatment 4), CBD was rapidly absorbed with median Tₘₐₓ ranged from 1.00 to 1.75 hours. The longer Tₘₐₓ observed in Treatment 2 was due to the late Tₘₐₓ (24 hour) observed in Animal 1. Following Tₘₐₓ, CBD plasma concentrations steadily declined with mean t1/2 ranging from 3.58 to 6.41 hours. The mean Cₘₐₓ ranged from 95.55 to 256.91 ng/mL and the mean AUCₗₐₛₜ ranged from 276.40 hr*ng/mL to 1139.02 hr*ng/mL The highest mean Cₘₐₓ (256.91 ng/mL) and AUCₗₐₛₜ (1139.02 hr*ng/mL) were observed in Treatment 4 (Vitamin E TPGS). The mean CBD Cₘₐₓ and AUCₗₐₛₜ observed in Treatment 4 were 2.5- and 4.1-fold higher than the mean values observed following the oral administration of CBD oil (Epidiolex) Treatment 1. The mean CBD Cₘₐₓ observed in Treatments 2 (95.55 ng/mL) and 3 (124.02 ng/mL) were generally similar to the mean Cₘₐₓ in Treatment 1 (102.93 ng/mL), while the mean AUCₗₐₛₜ values (559.89 hr*ng/mL and 540.1 hr*ng/mL, respectively, for Treatments 2 and 3) were approximately 2-fold higher than the mean AUCₗₐₛₜ for Treatment 1 (276.4 hr*ng/mL).

The highest mean Cₘₐₓ and AUCₗₐₛₜ were observed in Treatment 4 (Vit E TPGS). The mean CBD Cₘₐₓ and AUCₗₐₛₜ observed in Treatment 4 were 2.5- and 4.1-fold higher than the mean values observed following oral administration of CBD oil (Epidiolex) Treatment 1.

### Example 3

### Pharmacokinetic Study

A randomised open label, two-way crossover, “fed” study comparing the Pharmacokinetic (PK) characteristics of one 150 mg dose of EMD-RX5 Cannabidiol (CBD) capsules with one 150 mg dose of Epidyolex™ CBD oil (100 mg/mL) in 12 healthy male and female volunteers aged 18-65.

The study comprised two dosing periods of 12 participants. In the first period, six participants were randomised to receive a single dose of 150 mg EMD-RX5 CBD capsules. The other six participants were randomised to receive a single dose of 150 mg of Epidyolex™ oil. During this time, participants were monitored for safety and PK samples were taken at scheduled intervals. Following a washout period of 7 days, participants returned to the clinic for the second treatment period. During the second dosing period, participants who received the Epidyolex™ oil in the first dosing period received EMD-RX5 CBD capsules, and those that received the EMD-RX5 capsules in the first dosing period received the oil in the second dosing period. Participants were again monitored for safety. Prior to all dosing, participants ate a high fat meal within 1 hour of taking their dose orally. PK samples were taken at set intervals for 24 hours from the time of study drug administration in each dosing period. 5-7 days after the second dosing period, participants returned to the clinical study site for a final safety follow up. During this visit vital signs were recorded, and blood taken for clinical laboratory parameters (haematology and chemistry). Adverse events were also recorded.

Healthy volunteers were screened for eligibility and only those meeting all inclusion/exclusion criteria were offered study participation. Written informed consent was obtained from each participant prior to performing screening procedures.

### Inclusion Criteria:

1. Adult males or females age 18-65 years, inclusive, with a body mass index 18-32 kg/m².
2. Blood glucose (random non-fasting), haemoglobin and platelets within normal limits at study entry as per site local laboratory ranges.

### Exclusion Criteria:

1. Poorly managed hypertension (systolic >160 mm Hg and/or diastolic >95 mm Hg) or hypotension (systolic 14 days prior to dosing.
2. Presence of any medical condition that results in cognitive deficits (e.g. active seizure disorder, stroke, vascular dementia)
3. History of major psychiatric illness or untreated depression as determined by investigator judgement.
3. History of drug or alcohol abuse within 6 months of screening or a positive alcohol breath test or urine drug screen.
4. Recreational or medicinal Cannabis drug use within 30 days of treatment.
5. Blood creatinine, aspartate aminotransferase (AST), alanine aminotransferase (ALT), alkaline phosphatase (ALP), gamma glutamyl transferase (GGT) or total bilirubin (TBL) more than 1.5 times above the Upper Limit of Normal (ULN) according to local laboratory ranges.
6. Inability to consume high fat meals due to allergies, intolerances or dietary preferences. (High fat meals are likely to include meat, animal products and/or gluten).

### Objectives

To describe the pharmacokinetic parameters of EMD-RX5 CBD 50 mg capsules after a once daily administration of 150 mg. Measurements included plasma CBD PK parameters Cₘₐₓ; Tₘₐₓ; AUC₀₋₂₄ hr; AUC_{inf}; T_{1/2} at the following times: pre-dose, 30 mins, 1 hour, 2 hours, 3 hours, 4 hours, 5 hours, 6 hours, 8 hours, 12 hours and 24 hours post dose.

Comparing the pharmacokinetic parameters of EMD-RX5 CBD 50 mg capsules after a single dose of 150 mg to a single dose of 150 mg of Epidyolex™ CBD oil.

Evaluating the safety and tolerability (Adverse Events) of EMD-RX5 CBD 50 mg capsules as assessed by standard clinical and laboratory measures.

To compare the parameters of metabolites 7-OH—CBD and 7-COOH—CBD, comparing a single dose of EMD-RX5 CBD 150 mg (3×50 mg capsules) to a single dose of 150 mg of Epidyolex™ CBD oil.

### Statistical Methods/Analysis

The analysis of the relative bioavailability between the two different formulations (EMD-RX5 capsule as test and Epidyolex™ oil as reference) was based on the dose-dependent plasma CBD parameters AUC_{inf}, AUC₀₋₂₄ hr and Cₘₐₓ. The natural log-transformed parameters were analysed separately using a mixed effect model with the treatment group (formulation), sequence and period as fixed effects and subject nested within sequence as a random effect. The geometric means (exponentiated least squares means) and the associated 90% confidence intervals for each treatment group and the geometric mean ratio (exponentiated difference between EMD-RX5 capsule least-squares mean as test and Epidyolex oil least-squares mean as reference) and the associated 90% confidence interval are presented. The geometric mean ratios and the associated confidence intervals are expressed as percentages.

Bioequivalence was concluded if the 2-sided 90% CI was completely contained within the interval (0.80, 1.25). The within- and between-subject variability of both treatments was estimated directly from the model for all AUC parameters and Cₘₐₓ.

The analysis was repeated based on the dose-normalised AUC_{inf}, AUC₀₋₂₄ hr and Cₘₐₓ results. Tₘₐₓ was analysed nonparametrically using the Wilcoxon signed rank test.

### Results

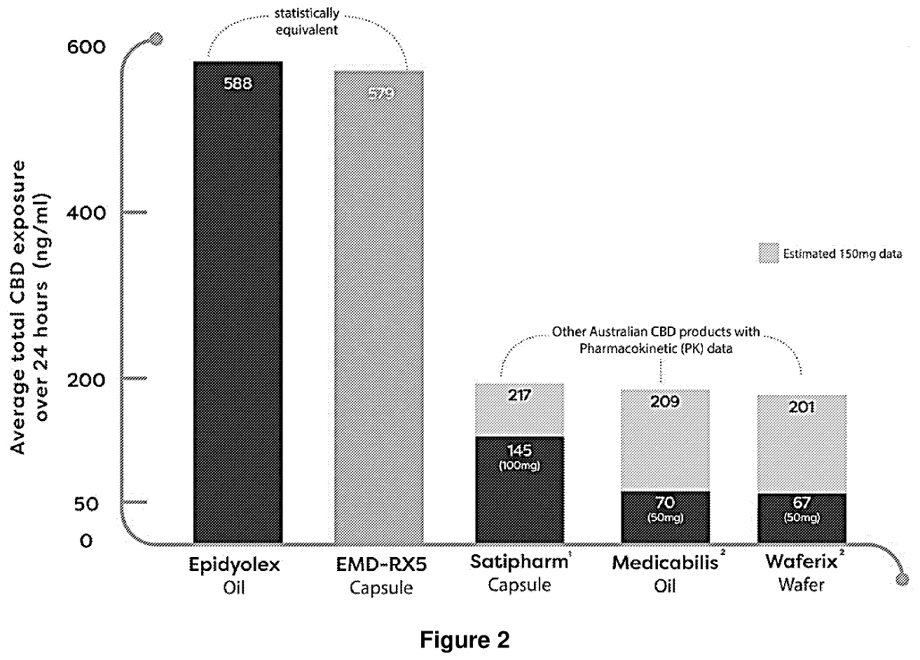

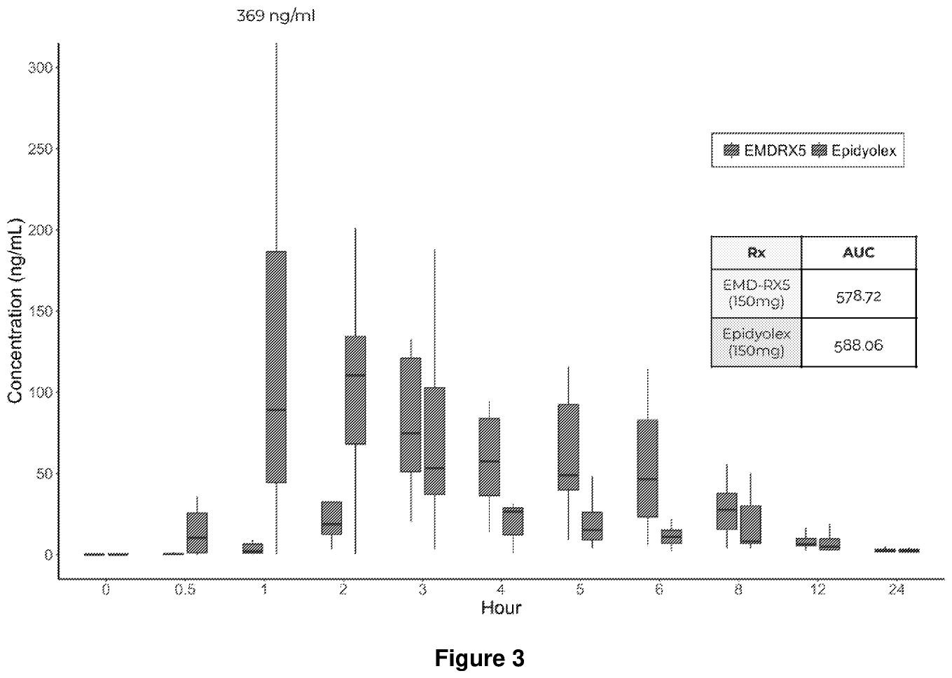

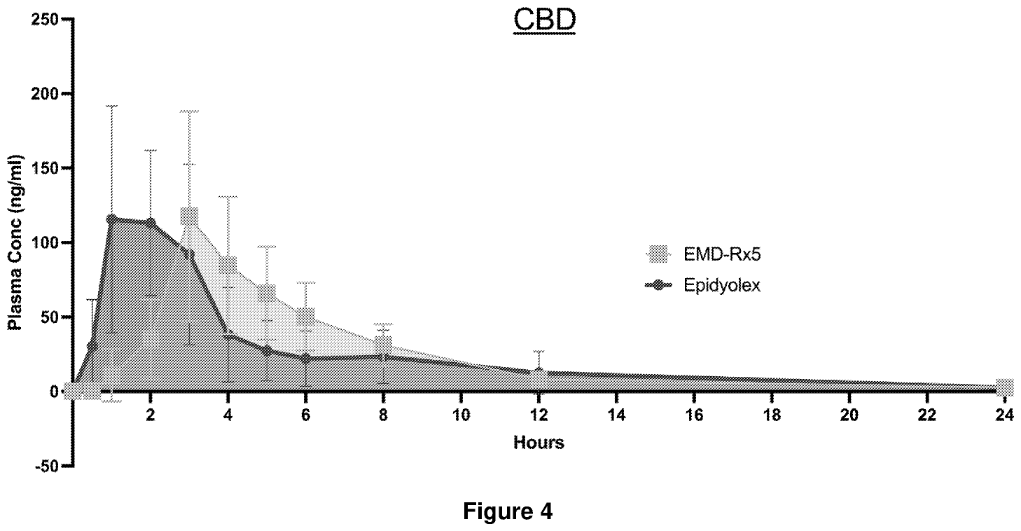

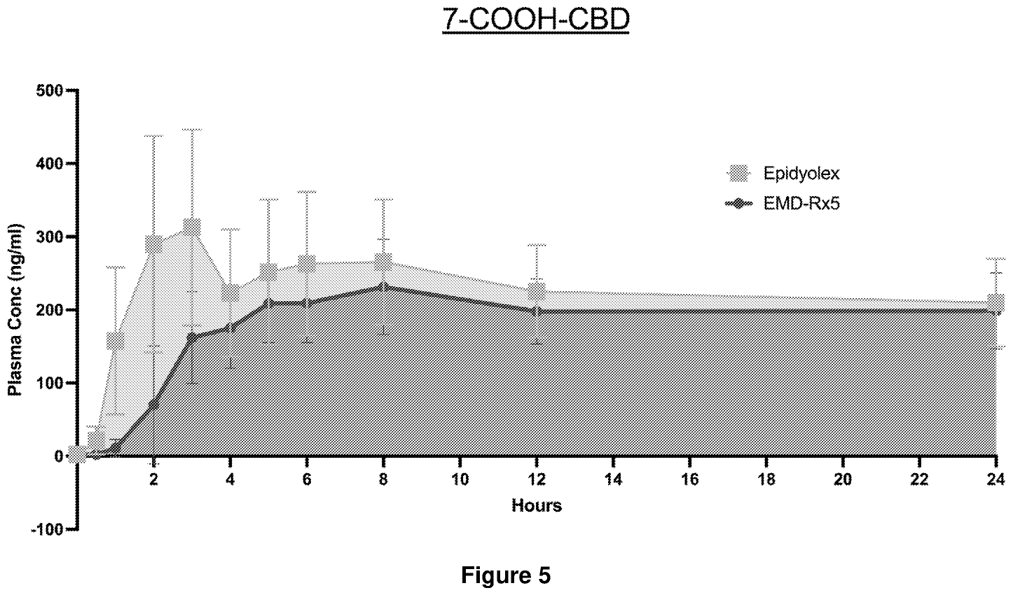

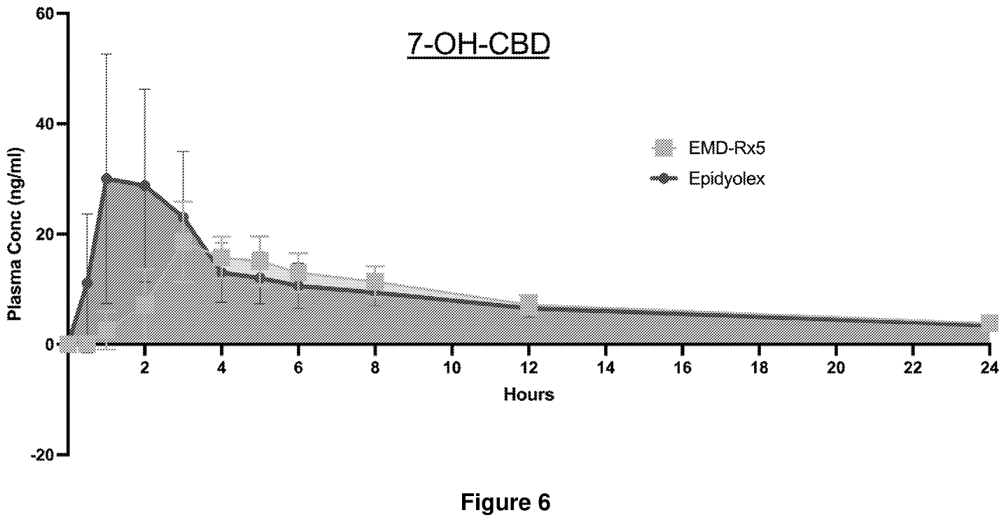

The results of the study are provided in Table 9 and FIGS. 2-6. Table 9 displays summary statistics for the recorded CBD concentration levels by EMD-RX5 and Epidyolex™. For each treatment the (i) mean total area under the curve (AUC), (ii) mean maximum concentration (Cₘₐₓ), and (iii) mean (and median) time at which a maximum peak was measured (Tₘₐₓ) were computed. The corresponding standard deviations (SD), standard errors (SE), and 95% confidence intervals (CI) are also shown. To assess bioequivalence for each measure (i.e., AUC, Cₘₐₓ, and Tₘₐₓ) the 90% confidence interval for the means of the log-transformed concentration levels were also calculated. The derived exponents (antilogs) of the confidence limits denote the 90% confidence interval for the ratio of the geometric means between the test and reference treatments. (i.e., EMD-RX5 and Epidyolex™).

TABLE 9
Preliminary analysis of PK metrics

| | | | | | Relative Bioequivalence | | |
|---|---|---|---|---|---|---|---|
| | PK Metrics | | | | (EMD-RX5/Epidyolex) 90% CI | | |
| Rx | AUC₀₋₂₄ | | | | Lower | AUC₀₋₂₄ | Upper |
| | (hr-ng/mL) | SD | SE | CI | limit | (hr-ng/mL) | limit |
| EMD-RX5 | 578.72 | 271.62 | 78.41 | 172.58 | 0.82 | O.96 | 1.12 |
| (150 mg) | | | | | | | |
| Epidyolex | 588.O6 | 228.47 | 65.95 | 145.16 | | | |
| (150 mg) | | | | | | | |
| | Cmax | | | | Lower | Cmax | Upper |
| | (ng/ml) | SD | SE | CI | limit | (ng/ml) | limit |
| EMD-RX5 | 129.08 | 76.86 | 22.19 | 48.84 | 0.48 | 0.70 | 1.01 |
| (150 mg) | | | | | | | |
| Epidyolex | 180.61 | 96.03 | 2772 | 61.02 | | | |
| (150 mg) | | | | | | | |
| | Tmax | | | | Lower | Tmax | Upper |
| | (median; hr) | SD | SE | CI | limit | (median; hr) | limit |
| EMD-RX5 | 3.75(300) | 1.29 | 0.37 | 0.82 | 0.88 | 1.39 | 2.18 |
| (150 mg) | | | | | | | |
| Epidyolex | 3.58(3.00) | 340 | 0.98 | 2.16 | | | |
| (150 mg) | | | | | | | |

### Example 3

### Cannabidiol Formulations

TABLE 10
Exemplary formulations of CBD in capsules

| CBD | CBD | Capsule | Gelucire | VitE/ | Total | | x-fold | total fill |
|---|---|---|---|---|---|---|---|---|
| [mg] | conc w/w | size | 50/13 [mg] | TPGS [mg] | excip [mg] | Excip % | Excip | [mg] |
| 50 | 16.7% | 2 | 250 | | 250 | 83% | 5 | 300 |
| 50 | 16.7% | 2 | | 250 | 250 | 83% | 5 | 300 |
| 100 | 16.7% | 0 | 500 | | 500 | 83% | 5 | 600 |
| 100 | 16.7% | 0 | | 500 | 500 | 83% | 5 | 600 |
| 100 | 25.0% | 1 | 300 | | 300 | 75% | 3 | 400 |
| 100 | 25.0% | 1 | | 300 | 300 | 75% | 3 | 400 |
| 100 | 25.0% | 1 | 270 | 30 | 300 | 75% | 3 | 400 |
| 100 | 25.0% | 1 | 240 | 60 | 300 | 75% | 3 | 400 |
| 100 | 33.3% | 2 | 200 | | 200 | 67% | 2 | 300 |
| 100 | 33.3% | 2 | | 200 | 200 | 67% | 2 | 300 |
| 100 | 33.3% | 2 | 180 | 20 | 200 | 67% | 2 | 300 |
| 100 | 33.3% | 2 | 160 | 40 | 200 | 67% | 2 | 300 |
| 150 | 25.0% | 0 | 450 | | 450 | 75% | 3 | 600 |
| 150 | 25.0% | 0 | | 450 | 450 | 75% | 3 | 600 |
| 150 | 25.0% | 0 | 405 | 45 | 450 | 75% | 3 | 600 |
| 150 | 25.0% | 0 | 427.5 | 22.5 | 450 | 75% | 3 | 600 |
| 150 | 37.5% | 1 | 250 | | 250 | 63% | 1.7 | 400 |
| 150 | 37.5% | 1 | | 250 | 250 | 63% | 1.7 | 400 |
| 150 | 37.5% | 1 | 225 | 25 | 250 | 63% | 1.7 | 400 |
| 150 | 37.5% | 1 | 200 | 50 | 250 | 63% | 1.7 | 400 |
| 200 | 33.3% | 0 | 400 | | 400 | 67% | 2 | 600 |
| 200 | 33.3% | 0 | | 400 | 400 | 67% | 2 | 600 |
| 200 | 33.3% | 0 | 360 | 40 | 400 | 67% | 2 | 600 |
| 200 | 33.3% | 0 | 320 | 80 | 400 | 67% | 2 | 600 |
| 200 | 40.0% | 0 | 300 | | 300 | 60% | 1.5 | 500 |
| 200 | 40.0% | 0 | | 300 | 300 | 60% | 1.5 | 500 |
| 200 | 40.0% | 0 | 270 | 30 | 300 | 60% | 1.5 | 500 |
| 200 | 40.0% | 0 | 240 | 60 | 300 | 60% | 1.5 | 500 |
| 200 | 45.5% | 1 | 240 | | 240 | 55% | 1.2 | 440 |
| 200 | 45.5% | 1 | | 240 | 240 | 55% | 1.2 | 440 |
| 200 | 45.5% | 1 | 216 | 24 | 240 | 55% | 1.2 | 440 |
| 200 | 45.5% | 1 | 192 | 48 | 240 | 55% | 1.2 | 440 |
| 250 | 41.7% | 1 | 350 | | 350 | 58% | 1.4 | 600 |
| 250 | 41.7% | 1 | | 350 | 350 | 58% | 1.4 | 600 |
| 250 | 41.7% | 1 | 315 | 35 | 350 | 58% | 1.4 | 600 |
| 250 | 41.7% | 1 | 280 | 70 | 350 | 58% | 1.4 | 600 |

TABLE 11
BHA and BHT levels for capsules with 200 mg CBD (allowing
for a maximum dose of four capsules per day)

| | | | Conc. for | Ratio | mg per | Conc. | Ratio |
|---|---|---|---|---|---|---|---|
| | MDE | mg per | 600 mg | BHT/ | caps | in | BHT/ |
| | [mg] | capsule | fill | A:CBD | RX5/7 | RX5/7 | A:CBD |
| BHT | 1 | 0.25 | 0.04% | 0.13% | 0.7 | 0.23% | 1.40% |
| BHA | 4 | 1 | 0.17% | 0.50% | 0.175 | 0.06% | 0.35% |

BHA and BHT levels can be adjusted for capsules with 100, 150 and 250 mg CBD per capsule depending on the intended maximal daily dose.

TABLE 12
Exemplary formulations of CBD in capsules

| mg/capsule | Component | % w/w | |
|---|---|---|---|
| 249.1 | Excipient (Gelucire 50/13 or | 83.0% | |
| | Vit E TPGS) | | |
| 0.7 | Butylated hydroxytoluene (BHT) | 0.2% | 0.25 mg |
| 0.175 | Butylated hydroxyanisole (BHA) | 0.1% | 1 mg |
| 50 | Cannabidiol (CBD) | 16.7% | |
| 300.0 | | | |

This formulation can be filled into size 2 gelatine hard capsules.

1. A pharmaceutical composition comprising:
a) a cannabinoid; and
b) a polyethylene glycol (PEG) ester.

2. The pharmaceutical composition of claim 1 that does not compromise an oil or lipid, or only compromises a minimal amount of an oil or lipid.

3. The pharmaceutical composition of claim 1 that comprises a ratio of from 1:5 w/w cannabinoid:PEG ester to 1:1.5 w/w cannabinoid:PEG ester.

4. A pharmaceutical composition comprising:
a) a cannabinoid; and
b) a polyethylene glycol (PEG) ester
wherein the bioavailability of the pharmaceutical composition comprising a polyethylene glycol (PEG) ester is from 0.8 to 12 times higher than the bioavailability of an oral pharmaceutical composition which comprises the same amount of cannabinoid but does not comprise a polyethylene glycol (PEG) ester.

5. A pharmaceutical composition comprising:
a) a cannabinoid; and
b) a polyethylene glycol (PEG) ester
wherein the prophylactically or therapeutically effective amount of the pharmaceutical composition comprising a polyethylene glycol (PEG) ester is from 0.08 to 1.25 times the prophylactically or therapeutically effective amount of an oral pharmaceutical composition which comprises the same amount of cannabinoid but does not comprise a polyethylene glycol (PEG) ester.

6. The pharmaceutical composition of claim 4 or 5 wherein the bioavailability is measured using:
AUC₀₋₂₄ and the AUC₀₋₂₄ of a cannabinoid in a composition of the present invention is from 0.8 to 12 times higher compared to the AUC₀₋₂₄ of a cannabinoid in a composition that does not comprise a PEG ester;
Tₘₐₓ and a cannabinoid in a composition of the present invention reaches the same Tₘₐₓ as cannabinoid in a composition that does not comprise a PEG ester from 0.5 to 2 times earlier than the Tₘₐₓ of the comparator composition;
Cₘₐₓ and the Cₘₐₓ of a cannabinoid in a composition of the present invention is from 0.8 to 12 times higher compared to the Cₘₐₓ of a cannabinoid in a composition that does not comprise a PEG ester; and/or
AUCₗₐₛₜ and the AUCₗₐₛₜ of a cannabinoid in a composition of the present invention is from 0.8 to 12 times higher compared to the AUCₗₐₛₜ of a cannabinoid in a composition that does not comprise a PEG ester.

7. The pharmaceutical composition of claim 4 or 5 wherein the cannabinoid has:
an AUC₀₋₂₄ of from 250 hr*ng/mL to 2000 hr*ng/mL;
a Tₘₐₓ of from 1.2 h to 2 h;
a Cₘₐₓ of from 40 ng/mL to 400 ng/mL; and/or
an AUCₗₐₛₜ of from 500 hr*ng/mL to 1,400 hr*ng/mL.

8. The pharmaceutical composition of any of claims 1 to 7 that is an oral pharmaceutical composition.

9. The pharmaceutical composition of any of claims 1 to 8 wherein the cannabinoid is chosen from the list comprising: cannabidiol, cannabinol, cannabigerol, cannabichromene, and Δ⁹-tetrahydrocannabinol. Most preferably, the cannabinoid is cannabidiol.

10. The pharmaceutical composition of any of claims 1 to 9 wherein the polyethylene glycol (PEG) ester is a stearoyl or lauroyl macrogol-32 glyceride or vitamin E polyethylene glycol succinate.

11. A method for the treatment, amelioration or prevention of a condition responsive to administration of a cannabinoid, said method comprising the step of:
a) administering a pharmaceutical composition comprising:
i. a cannabinoid;
ii. a polyethylene glycol (PEG) ester.

12. A method for the treatment, amelioration or prevention of a condition responsive to administration of a cannabinoid, said method comprising the step of:
a) administering a pharmaceutical composition comprising:
i. a cannabinoid;
ii. a polyethylene glycol (PEG) ester
wherein the bioavailability of the pharmaceutical composition comprising a polyethylene glycol (PEG) ester is from 0.8 to 12 times higher than the bioavailability of an oral pharmaceutical composition which comprises the same amount of cannabinoid but does not comprise a polyethylene glycol (PEG) ester.

13. A method for the treatment, amelioration or prevention of a condition responsive to administration of a cannabinoid, said method comprising the step of:
a) administering a pharmaceutical composition comprising:
i. a cannabinoid;
ii. a polyethylene glycol (PEG) ester
wherein the prophylactically or therapeutically effective amount of the pharmaceutical composition comprising a polyethylene glycol (PEG) ester is from 0.08 to 1.25 times the prophylactically or therapeutically effective amount of an oral pharmaceutical composition which comprises the same amount of cannabinoid but does not comprise a polyethylene glycol (PEG) ester.

14. Use of:
a) a cannabinoid; and
b) a polyethylene glycol (PEG) ester
for the manufacture of a pharmaceutical composition for the treatment, amelioration or prevention of a condition responsive to administration of a cannabinoid, wherein the

15. Use of:
a) a cannabinoid; and
b) a polyethylene glycol (PEG) ester
for the manufacture of a pharmaceutical composition for the treatment, amelioration or prevention of a condition responsive to administration of a cannabinoid, wherein the bioavailability of the pharmaceutical composition comprising a polyethylene glycol (PEG) ester is from 0.8 to 12 times higher than the bioavailability of an oral pharmaceutical composition which comprises the same amount of cannabinoid but does not comprise a polyethylene glycol (PEG) ester.

16. Use of:
a) a cannabinoid; and
b) a polyethylene glycol (PEG) ester
for the manufacture of a pharmaceutical composition for the treatment, amelioration or prevention of a condition responsive to administration of a cannabinoid, wherein the prophylactically or therapeutically effective amount of the pharmaceutical composition comprising a polyethylene glycol (PEG) ester is from 0.08 to 1.25 times the prophylactically or therapeutically effective amount of an oral pharmaceutical composition which comprises the same amount of cannabinoid but does not comprise a polyethylene glycol (PEG).

17. Use of a composition comprising:
a) a cannabinoid; and
b) a polyethylene glycol (PEG) ester
for the treatment, amelioration or prevention of a condition responsive to administration of a cannabinoid, wherein the

18. Use of a composition comprising:
a) a cannabinoid; and
b) a polyethylene glycol (PEG) ester
for the treatment, amelioration or prevention of a condition responsive to administration of a cannabinoid, wherein the bioavailability of the pharmaceutical composition comprising a polyethylene glycol (PEG) ester is from 0.8 to 12 times higher than the bioavailability of an oral pharmaceutical composition which comprises the same amount of cannabinoid but does not comprise a polyethylene glycol (PEG) ester.

19. Use of a composition comprising:
a) a cannabinoid; and
b) a polyethylene glycol (PEG) ester
for the treatment, amelioration or prevention of a condition responsive to administration of a cannabinoid, wherein the prophylactically or therapeutically effective amount of the pharmaceutical composition comprising a polyethylene glycol (PEG) ester is from 0.08 to 1.25 times the prophylactically or therapeutically effective amount of an oral pharmaceutical composition which comprises the same amount of cannabinoid but does not comprise a polyethylene glycol (PEG).

20. A kit for the treatment, amelioration or prevention of a condition responsive to administration of a cannabinoid, said kit comprising:
a) a pharmaceutical composition comprising:
i. a cannabinoid; and
ii. a polyethylene glycol (PEG) ester
b) instructions for use
wherein the bioavailability of the pharmaceutical composition comprising a polyethylene glycol (PEG) ester is from 0.8 to 12 times higher than the bioavailability of an oral pharmaceutical composition which comprises the same amount of cannabinoid but does not comprise a polyethylene glycol (PEG) ester.

21. A kit for the treatment, amelioration or prevention of a condition responsive to administration of a cannabinoid, said kit comprising:
a) a pharmaceutical composition comprising:
i. a cannabinoid; and
ii. a polyethylene glycol (PEG) ester
b) instructions for use.

22. A kit for the treatment, amelioration or prevention of a condition responsive to administration of a cannabinoid, said kit comprising:
a) a pharmaceutical composition comprising:
i. a cannabinoid; and
ii. a polyethylene glycol (PEG) ester
b) instructions for use
wherein the prophylactically or therapeutically effective amount of the pharmaceutical composition comprising a polyethylene glycol (PEG) ester is from 0.08 to 1.25 times the prophylactically or therapeutically effective amount of an oral pharmaceutical composition which comprises the same amount of cannabinoid but does not comprise a polyethylene glycol (PEG) ester.

23. The method of any of claims 11 to 13, use of any of claims 15 to 19, or kit of any of claims 20 to 22 that does not compromise an oil or lipid, or only compromises a minimal amount of an oil or lipid.

24. The method of any of claims 11 to 13, use of any of claims 15 to 19, or kit of any of claims 20 to 22 that comprises a ratio of from 1:5 w/w cannabinoid:PEG ester to 2:3 w/w cannabinoid:PEG ester.

25. The method of any of claims 11 to 13, use of any of claims 15 to 19, or kit of any of claims 20 to 22 wherein the bioavailability is measured using:
AUC₀₋₂₄ and the AUC₀₋₂₄ of a cannabinoid in a composition of the present invention is from 0.8 to 12 times higher compared to the AUC₀₋₂₄ of a cannabinoid in a composition that does not comprise a PEG ester;
AUC_{0-inf} of a cannabinoid in a composition of the present invention is from 0.08 to 1.25 times higher compared to the AUC_{0-inf} of a cannabinoid in a composition that does not comprise a PEG ester;
Tₘₐₓ and a cannabinoid in a composition of the present invention reaches the same Tₘₐₓ as cannabinoid in a composition that does not comprise a PEG ester from 0.5 to 2 times earlier than the Tₘₐₓ of the comparator composition;
Cₘₐₓ and the Cₘₐₓ of a cannabinoid in a composition of the present invention is from 0.8 to 12 times higher compared to the Cₘₐₓ of a cannabinoid in a composition that does not comprise a PEG ester; and/or
AUCₗₐₛₜ and the AUCₗₐₛₜ of a cannabinoid in a composition of the present invention is from 0.8 to 12 times higher compared to the AUCₗₐₛₜ of a cannabinoid in a composition that does not comprise a PEG ester.

26. The method of any of claims 11 to 13, use of any of claims 15 to 19, or kit of any of claims 20 to 22 wherein the cannabinoid has:
an AUC₀₋₂₄ of from 250 hr*ng/mL to 2000 hr*ng/mL;
AUC_{0-inf} of from 250 hr*ng/mL to 2,000 hr*ng/mL;
a Tₘₐₓ of from 1.2 h to 2 h;
a Cₘₐₓ of from 40 ng/mL to 400 ng/mL; and/or
an AUCₗₐₛₜ of from 500 hr*ng/mL to 1,400 hr*ng/mL.

27. The method of any of claims 11 to 13, use of any of claims 15 to 19, or kit of any of claims 20 to 22 wherein the pharmaceutical composition is an oral pharmaceutical composition.

28. The method of any of claims 11 to 13, use of any of claims 15 to 19, or kit of any of claims 20 to 22 wherein the cannabinoid is chosen from the list comprising: cannabidiol, cannabinol, cannabigerol, cannabichromene, and Δ⁹-tetrahydrocannabinol. Most preferably, the cannabinoid is cannabidiol.

29. The method of any of claims 11 to 13, use of any of claims 15 to 19, or kit of any of claims 20 to 22 wherein the polyethylene glycol (PEG) ester is a stearoyl or lauroyl macrogol-32 glyceride or vitamin E polyethylene glycol succinate.
